(19) United States
(12) Patent Application Publication
(10) Pub. No.: US 2026/0198846 A1
(43) Pub. Date: Jul. 16, 2026

(54) SYSTEM AND METHOD FOR CAPTURING HEALTH ANALYTICS WITH A MOBILE DEVICE

(71) Applicant: Spectricity, Mechelen (BE)

(72) Inventors: Maarten De Bock, Gent (BE); Robbe van Beers, Kessel (BE); Ruben Lieten, Heverlee (BE); Jakub Raczkowski, Bertem (BE); Peter van Wesemael, Antwerpen (BE); Jonathan Borremans, Lier (BE); Ward van der Tempel, Keerbergen (BE)

(73) Assignee: Spectricity, Mechelen (BE)

(21) Appl. No.: 19/563,047

(22) Filed: Mar. 11, 2026

Related U.S. Application Data
(63) Continuation of application No. 18/296,589, filed on Apr. 6, 2023.
Application No. 18/296,589 is a continuation of application No. PCT/US2021/053531, filed on Oct. 5, 2021.
(63) Continuation of application No. 19/405,011, filed on Dec. 1, 2025.
Application No. 19/405,011 is a continuation of application No. 16/959,631, filed on Jan. 22, 2019, now Pat. No. 12,484,836.
(60) Provisional application No. 63/088,542, filed on Oct. 7, 2020.

(30) Foreign Application Priority Data
Jan. 22, 2018 (EP) 18152787.0

(51) Int. Cl. A61B 5/00; G06T 7/00

(52) U.S. Cl. CPC A61B 5/441; A61B 5/0075; A61B 5/7264; G06T 7/0014

(57) ABSTRACT

A mobile device includes one or more interface and a plurality of spectrometers positioned in the mobile device to capture light radiation incident to the mobile device and output information representative of captured light radiation. The mobile device further includes a processing module adapted to receive output information and determine an accumulated light radiation for the mobile device and a notification engine adapted to generate an alert when the accumulated light radiation exceeds a predetermined threshold.

### CROSS-REFERENCE TO RELATED APPLICATIONS

The present application claims priority claims priority pursuant to 35 U.S.C. § 120 as a continuation of U.S. Utility application Ser. No. 18/296,589, entitled “HEALTH ANALYSIS USING A SPECTRAL SENSOR SYSTEM”, filed Apr. 6, 2023, which is a continuation of International Application Number PCT/US2021/053531, entitled “HEALTH ANALYSIS USING A SPECTRAL SENSOR SYSTEM’, filed Oct. 5, 2021, which claims priority pursuant to 35 U.S.C. § 119(e) to U.S. Provisional Patent Application No. 63/088,542 , entitled “HEALTH ANALYSIS USING A SPECTRAL SENSOR SYSTEM’, filed Oct. 7, 2020, all of which are incorporated herein by reference in their entirety and made part of the present application for all purposes.

The present application also claims priority pursuant to 35 U.S.C. § 120 as a continuation of U.S. Utility application Ser. No. 19/405,011, entitled “OPTICAL RESPONSE MEASUREMENT FROM SKIN AND TISSUE USING SPECTROSCOPY”, filed Dec. 1, 2025, which is a continuation of U.S. Utility application Ser. No. 16/959,631, entitled “OPTICAL RESPONSE MEASUREMENT FROM SKIN AND TISSUE USING SPECTROSCOPY”, filed Jul. 1, 2020, issued as U.S. Pat. No. 12,484,836 on Dec. 2, 2025, which claims priority pursuant to U.S.C. § 371 as a U.S. National Phase Application of International Application No. PCT/EP2019/051494, entitled “OPTICAL RESPONSE MEASUREMENT FROM SKIN AND TISSUE USING SPECTROSCOPY”, filed Jan. 22, 2019, which claims priority to EP Application No. 18152787.0, entitled “A WEARABLE DEVICE FOR AND METHODS RELATED TO THE MEASUREMENT OF THE OPTICAL RESPONSE OF SKIN AND UNDERLYING TISSUE”, filed Jan. 22, 2018, all of which are hereby incorporated herein by reference in their entirety and made part of the present U.S. Utility Patent Application for all purposes.

### BACKGROUND OF THE INVENTION

### Technical Field of the Invention

This invention relates generally to spectroscopy and more particularly to measuring physiological parameters related to health using optical spectroscopy.

Spectroscopy devices have proven to be useful for applications in various industries including, for example, health, biometrics, agriculture, chemistry and fitness. Spectroscopy involves the measurement of spectra produced when matter interacts with or emits electromagnetic radiation. Diffuse optical reflectance spectroscopy involves illuminating a material and detecting light from the material being illuminated. In the case of diffuse optical reflectance spectroscopy, propagated light from a material is captured at the detector, whereas transmittance spectroscopy involves the capture of light transmitted through the material at the detector. Interference-based filters, such as Fabry-Pérot filters, when used in conjunction with spectroscopy, have been shown to be capable of providing useful spectral information.

A light source penetrates material based on the components of the light source and the properties of the material and is captured by a detector as a combination of propagated, scattered and transmitted light that can reveal attributes of the material.

### BRIEF DESCRIPTION OF THE SEVERAL VIEWS OF THE DRAWING(S)

FIG. 1A provides a top-down illustration of an example spectral filter array in accordance with the present invention;

FIG. 1B provides a side-view illustration of an example optical sensor overlaid with filters in accordance with the present invention;

FIG. 2A illustrates a camera module for a mobile device incorporating an image sensor and a spectral sensor in accordance with the present invention;

FIG. 2B illustrates a camera module for a mobile device incorporating an image sensor, a spectral sensor and an illumination source in accordance with the present invention;

FIG. 2C is a block diagram of a camera module configuration for a mobile device incorporating a spectroscopy device in accordance with the present invention;

FIG. 3A is a flowchart illustrating an example method for determining a radiation exposure in accordance with the present invention;

FIG. 3B is a flowchart illustrating an example method for determining an accumulated radiation exposure in accordance with the present invention;

FIG. 3C is a flowchart illustrating an example method for classifying skin type in accordance with the present invention;

FIG. 4A illustrates a mobile device with a forward-facing camera module incorporating an image sensor, a spectral sensor and an illumination source in accordance with the present invention;

FIG. 4B illustrates a mobile device with a back-facing camera module incorporating an image sensor, a spectral sensor and an illumination source in accordance with the present invention;

FIG. 4C illustrates a mobile device with a forward-facing spectral sensor and a back facing spectral sensor in accordance with the present invention;

FIG. 4D illustrates a wrist mounted spectral sensor in accordance with the present invention;

FIG. 5A is a flowchart illustrating an example method for determining skin parameters in accordance with the present invention;

FIG. 5B is a flowchart illustrating an example method for detecting and classifying skin aberrations in accordance with the present invention;

FIG. 6A is a flowchart illustrating an example method for determining skin parameters using a spectral sensor in accordance with the present invention;

FIG. 6B is a flowchart illustrating another example method for determining skin parameters using a spectral sensor in accordance with the present invention;

FIG. 7A is a flowchart illustrating an example method for classifying skin type for use in providing skin treatment in accordance with the present invention;

FIG. 7B is a flowchart illustrating another example method for classifying skin type for use in providing skin treatment in accordance with the present invention;

FIG. 8 is a flowchart illustrating an example method for using body area parameters from spectral sensing for biometric analysis in accordance with the present invention;

FIG. 9 is a flowchart illustrating an example method for using the combined output from an image sensor and a spectral sensor in accordance with the present invention;

FIG. 10 is a flowchart illustrating an example method for determining applied pressure using a spectral sensor in accordance with the present invention;

FIG. 11A provides an illustration of a spectral sensing system incorporating multiple spectral sensors in accordance with the present invention;

FIG. 11B provides another illustration of a spectral sensing system incorporating multiple spectral sensors in accordance with the present invention;

FIG. 12A provides another illustration of a spectral sensing system incorporating multiple spectral sensors in accordance with the present invention;

FIG. 12B is a flowchart of a method for determining the spectrophotometric parameters of a material using multiple spectral sensors in accordance with the present invention;

FIG. 13A illustrates Isosbestic Points for a water absorption peak as a function of temperature;

FIG. 13B is a flowchart of a method for determining the temperature of skin or other tissue using a spectrophotometer in accordance with the present invention;

FIG. 14A is a flowchart of a method for collecting a photoplethysmogram using a spectrophotometer in accordance with the present invention;

FIG. 14B is a flowchart of a method for collecting a photoplethysmogram (PPG) using a spectrophotometer in accordance with the present invention;

FIG. 15A is a block diagram of a system for a measuring range incorporating a spectroscopy device in accordance with the present invention;

FIG. 15B is a flowchart of a method for determining time-of-flight using a spectrophotometer in accordance with the present invention;

FIG. 16 illustrates a system for monitoring blood pressure using multiple spectral sensors in accordance with the present invention;

FIG. 17 is a flowchart illustrating an example method for monitoring wound healing using a spectral sensor in accordance with the present invention;

FIG. 18 is a flowchart illustrating an example method for using a spectral sensor to augment other sensors in accordance with the present invention;

FIG. 19A provides an illustration of a spectral sensor system that uses photoplethysmogram (PPG) signals to determine sample parameters in accordance with the present invention;

FIG. 19B is a flowchart illustrating an example method for using a spectrophotometer to conform the validity of sample analysis in accordance with the present invention;

FIG. 19C is a flowchart illustrating another example method for using a spectrophotometer to confirm the validity of sample analysis in accordance with the present invention; and

FIG. 19D is a flowchart illustrating an example method for using a spectrophotometer to measure the water content of skin or tissue in accordance with the present invention.

### DETAILED DESCRIPTION OF THE INVENTION

In various embodiments, spectral image sensors are combined with spectral filters such as interference-based interference filters to provide spectral information about the health, fitness and safety of skin, tissue and environment. In some embodiments, spectral imaging of a material can be performed and in other embodiments spectral imaging of a scene can either be combined with high resolution imaging of an imaging device, or separate imagers combined after an image is collected. In further embodiments, interference-based filters can be implemented using Fabry-Perot filters integrated with spectral image sensors, such as CMOS-based sensors, to provide small-scale spectral image sensor systems. In some embodiments, small-scale spectral imaging systems can be adapted for use in mobile devices. Examples of mobile devices include, but are not limited to, smart mobile phones, smart watches, calibration devices, medical equipment, fitness devices and crowd-sourced monitoring devices.

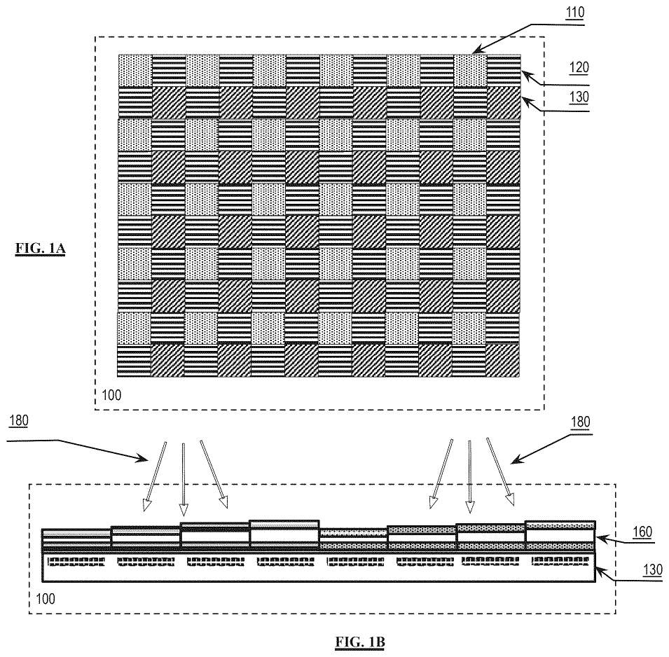

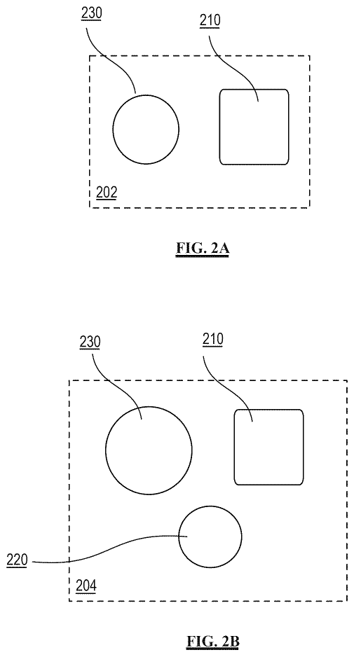

FIG. 1A provides a top-down illustration of an integrated spectral filter array 100 overlaid with filters 110, 120 and 130, each optimized for one of three spectral bands, respectively. As shown filters 110, 120 and 130 repeat as an array across the surface of spectral filter array 100. In an example (not shown) filters for spectral bands exceeding 3 could be used to overlay sensors as desired in any practical orientation, with the spectral bands combining to provide a spectrum of wavelengths. FIG. 1B provides a side-view illustration of an example optical sensor overlaid with a filter array. In an example, incident light 180 is directed to optical sensor array 130 through filter array 160 (for example, the repeating filters 110, 120 and 130 of FIG. 1A). In an embodiment, spectral sensor 100 is an example of a spectral sensor useful for diffuse optical spectroscopy, where arrays of spectral filters are associated with optical sensors to provide diffuse spectral sensing.

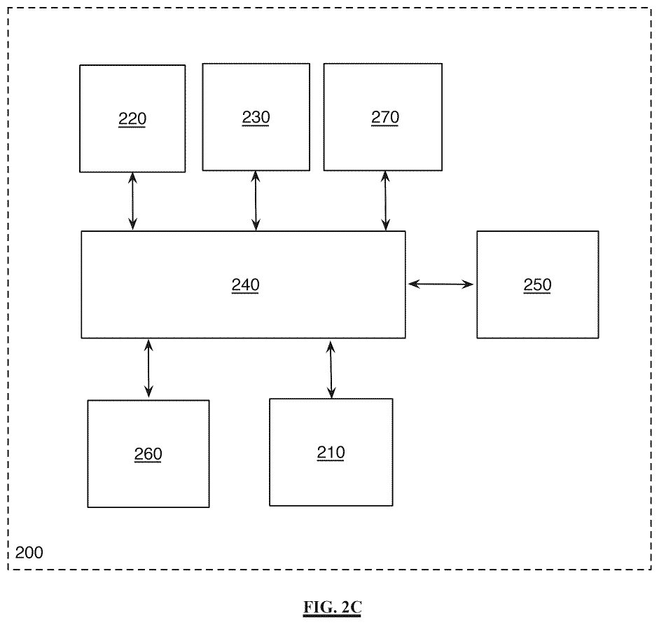

FIG. 2A illustrates a camera module 202 for a mobile device incorporating an image sensor 230 and a spectral sensor 210. In an example, spectral sensor 210 is configured to provide a spectral information about an object or scene, while image sensor 230 is configured to provide an image of the same object or scene. In an example, the response from spectral sensor 210 can be used to provide spectral information for spatial areas of an object imaged with the image sensor 230.

FIG. 2B illustrates the mobile device of FIG. 2A further incorporating a camera module 202 that includes illumination source 220. In an example, the illumination source 220 provides light in a predetermined range of optical wavelengths and is configured to irradiate light directly onto an object, with the spectral sensor 210 having a sensing range substantially matched to the predetermined range of optical wavelengths and configured to directly capture light emitted from the object, whereby the spectral sensor 210 is positioned a predetermined distance from the illumination source 220 to capture irradiated light when being emitted from the object.

Referring to FIGS. 2A and 2B, diffuse optical reflectance or transmittance spectroscopy consists of illuminating an object, such as skin or tissue with an illumination source, such as illumination source 220 or with natural light or a combination of both and using a suitable detector to capture light (propagated light in case of reflectance spectroscopy, transmitted light in case of transmittance spectroscopy, or a combination thereof). At a first level, light incident on an object surface penetrates the object interior (such as the tissue underneath skin) and is scattered, propagated or absorbed by the tissue depending on the relevant properties of the object. A spectral sensor 210 can be used to collect light that has passed through an object, such as tissue in the case of transmittance or transmissive spectrometry, In an example, the spectral sensor 210 can also be used to collect light that has propagated from constituents included in an object or tissue, where the light collected can be a result of both transmitted light and propagated light, such that the light collected is a function of the trans-reflective properties of the object or tissue.

At a second level of operation and implementation, incident light penetrating an object or tissue can induce complex interactions with the object or tissue, such as Raman scattering, where inelastic scattering of photons by an object or tissue's constituents can result from an exchange of energy and a change in the light's direction. In the case of Raman scattering, this can involve vibrational energy being gained by a molecule as incident photons from a light source are shifted to lower energy. Other examples include Black-body radiation, where light source induced heat can result in the output of a specific spectrum of wavelengths, inversely related to intensity that depends only on the body's temperature. In each case, the extent of penetration of light into the object or tissue depends on the wavelength components in the light source relative to the object properties. Thus, the light captured by a detector is a mix of light which has been propagated, scattered and transmitted by the illuminated object and its components (such as layers, tissues, blood vessels, etc. of skin). For the purposes of this document, propagated, scattered and transmitted light received from an illuminated object (whether by an illumination source or from natural light) at a detector are collectively considered to have been propagated by the object (such as skin or other tissue). In an example, a predetermined illumination distance can be selected to match a desired penetration path of the irradiated light having a predetermined wavelength.

FIG. 2C is a block diagram of a configuration for a camera module 200 for a mobile device incorporation a spectroscopy device in accordance with the present invention. In an example, mobile device camera module 200 can comprise or more spectral sensors 210. In an example, spectral sensors 210 can incorporate interference-based filters such as, for example, Fabry-Pérot filters. Other types of interference-based filters, such as thin film filters or plasmonic filters, can be used, along with noninterference-based, either alone or in combination. In an example, spectral sensors 210 can be CMOS imager sensors, non-CMOS based optical sensors that can be used to extend the spectral range of a spectral sensor to infrared wavelengths and pinned photodiodes. For example, colloidal or quantum dot-based optical sensor may be used to collect infrared light, for example in the short-wave infrared range. In the example of a quantum dot-based optical sensor, the optical sensors may be optimized by tuning the quantum dot size, such that a predefined wavelength is selected, so that the optical sensor provides an infrared filter channel. In the example of pinned photodiodes, a “pinned photodiode” is a photodetector structure available in charge-coupled device (CCD) and CMOS image sensors. The pinned photodiode includes a “buried” P/N junction that is physically separated from a sensor substrate, such that applying an appropriate bias depletes the P/N junction of electrons, allowing it to provide a nearly perfect “dark” pixel response, along with low noise, high quantum efficiency, low lag and low dark current.

In particular spectroscopy implementations, pinned photodiodes can provide high sensitivity, which is ideal to detect an attenuated signal remaining after light from the illumination source has interacted with, for example, skin or tissue. The attenuation can be due to absorption and scattering of light inside the skin. In addition to their other attributes detailed above, pinned photodiodes can provide fast response, allowing the sampling signals in the hundreds of hertz (Hz), which can be advantageous, for example, in photoplethysmogram (PPG) measurements or heart rate monitoring. The fast response of pinned diodes is a result of the high sensitivity of the pinned photodiodes, which allows short integration times. In an example, the high sensitivity of pinned photodiodes can help mitigate light transmission due to the spectral filters on the spectral sensor, which, because of optical filtering, significantly attenuate light received at optical sensors. For example, when a spectral sensor operates on 128 spectral channels, the optical area is reduced by 12833 per channel, therefore reducing sensitivity of the spectral sensor by a commensurate amount. Accordingly, collecting 128 PPG signals can benefit immensely from increased sensitivity associated with a highly sensitive detector, such as a pinned photodiode, single-photon avalanche detector (SPAD) or avalanche photo-detector (APD).

One or more illumination sources 220 can comprise one or more Light Emitting Diodes (LEDs) or Vertical Cavity Surface Emitting LEDs (VSCELs) as desired to provide wavelengths of interest. Illumination sources 220 may also contain one or more LEDs with phosphor coatings to extend the spectral range of the LED. In an example, the LEDs can contain a combination of wideband (phosphor-based) LEDs and narrow-band LEDs. Illumination sources 220 can also include other light sources, such as illumination sources adapted to provide wavelengths in the near-infrared (NIR), infrared (IR) and ultraviolet (UV) light spectrums.

Memory 250 can be included to store collected data and/or instructions. Depending on a type of apparatus in which one or more spectral sensors 210 are implemented, the memory can either be dedicated for spectral sensors 210 or shared with other functionalities of the mobile phone and or camera module 200. In an embodiment, memory 250 can contain instructions for executing a chemometric algorithm for deriving one or more physiological parameters influencing the irradiated light. In another embodiment, the memory stores specific calibration parameters related to the spectral sensors 210, such as, for example, its illumination or optics. In yet another embodiment, the memory can store specific biometric data of a user.

In an example, one or more batteries 260 can be included to power spectral sensors 210 and can be dedicated or shared with other camera module functions and/or spectral and image processing. Battery 260 can be one-time chargeable or rechargeable. In an example when battery 260 is rechargeable it can be charged either wirelessly or through a wired connection. Computing device 240 can be configured to process and manage the collection of data acquired from spectral sensors 210, and can be dedicated to spectrophotometric functions or shared for image sensor and/or mobile device functions. In a specific example, all or a portion of the elements of mobile device 200 can be configured to communicate wirelessly with the mobile device 200. In a related example, one or more wireless connectivity devices associated with a mobile device can be configured to communicate with the camera module 200, including one or more of image sensor 230 and/or additional sensors 270, any of which can themselves be configured to communicate wirelessly with the mobile device. In a related example, mobile device can be configured to manage connectivity between one or more sensors adapted for communication with camera module 200. In yet another example, a plurality of sensors configured be configured to communicate as a mesh network with mobile device 200 and in a related example, a plurality of connected sensors can comprise a body area network with sensors distributed on a user's body.

One or more additional sensors 270 can be included. Examples of such other sensors include EKG sensors, inertial measurement unit (IMU) sensors, electrical impedance sensors, skin temperature sensors or any other sensor that can be used to obtain other sensory information to correlate to or complement collected spectral data. The camera module 200 can include additional functions/modules, (not shown) such as one or more range computing modules, and one or more control circuits.

In an example, a spectral sensor 210 is used to measure a radiation level of an environment over a spectrum of wavelengths. Adequate exposure to light radiation such as sunlight is known to be important for overall health and prevention from disease, while too much exposure to light radiation can be harmful to health. For example, ultraviolet (UV) radiation is classified according to wavelength: UVA (longest wavelength), UVB (medium wavelength), and UVC (shortest wavelength). Appropriate exposure to sunlight and specifically to UVB radiation is necessary for the production of vitamin D, but at the same time an excessive exposure to other UV radiation wavelengths, such as UVC can increase the risk of developing certain health conditions such as skin cancer.

In an example of implementation, radiation exposure, such as ultraviolet (UV) radiation exposure can be monitored in an environment encountered by a mobile device user. The illumination conditions around an individual can be monitored, while information related to proactive preventative measures, such as preferred exposure time or informing dosing of sunscreen or other protection products, can be provided to the individual to optimize exposure to the light radiation. Examples include UV radiation from various sources, such as direct sunlight, UV lamps, tanning beds and incidental UV sources encountered in personal and industrial settings. In an example, the attenuation and/or amplification of UV in different environments, such as outdoor environments with cloud cover (or other weather-related conditions) can be monitored to enable a mobile device user to be notified if predetermined thresholds of instantaneous and/or accumulated radiation are exceeded. In an example a spectral sensor can be configured to provide a spectral response to near infrared (NIR), middle infrared (MIR), ultraviolet (UV) radiation, along with the full spectrum of visible light radiation.

In a specific example of implementation and operation, a mobile device includes one or more interfaces, with one or more spectrometers operably coupled to an interface, where each of the one or more spectrometers includes a plurality of spectral filters overlaying one or more optical sensors. In an example, each of the one or more spectrometers has a sensing range within a predetermined range of optical wavelengths and the one or more spectrometers are positioned in the mobile device to capture radiation incident to a user and are adapted to output information representative of captured radiation over the interface. In an example, the mobile device includes a local memory and a processing module operably coupled to the one or more interfaces and the local memory; the processing module being adapted to receive the output information representative of captured radiation and determine a total radiation incident to the mobile device.

In an example of implementation, a notification engine is included and is adapted to signal a user of the mobile device when the total radiation exceeds a predetermined threshold. In an additional example, the processing module is adapted to determine an accumulated total radiation over a period of time T, and in a related example, the accumulated radiation is determined over a spectrum of wavelengths. In yet another example, a mobile device user can manually determine the start of time T and in another example, the time T is determined based on external indicia, such as location, temperature, a change in measured radiation, etc. In an example, total the radiation can be determined on comparison to a predetermined spectral profile.

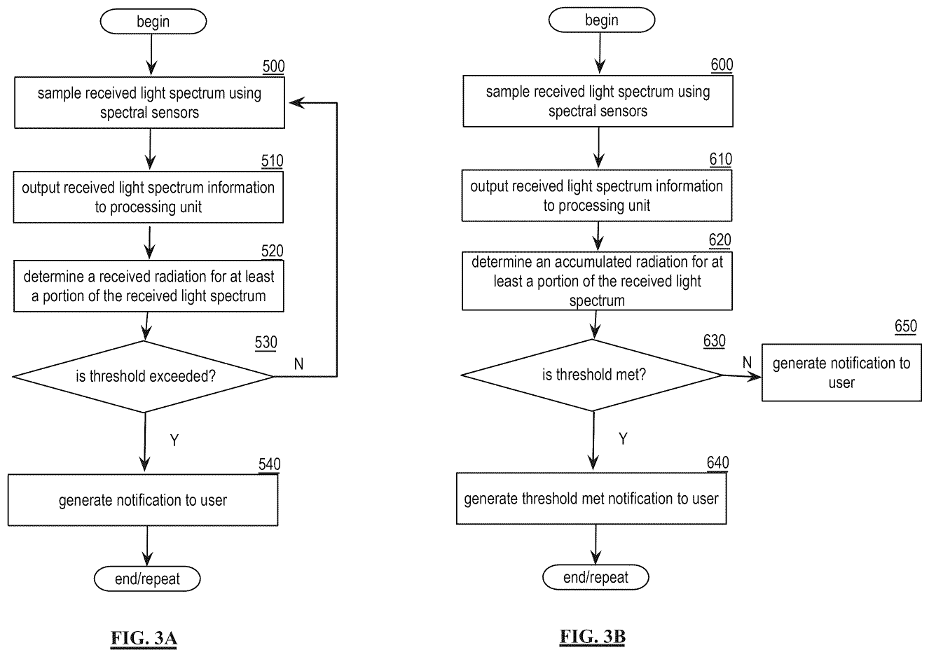

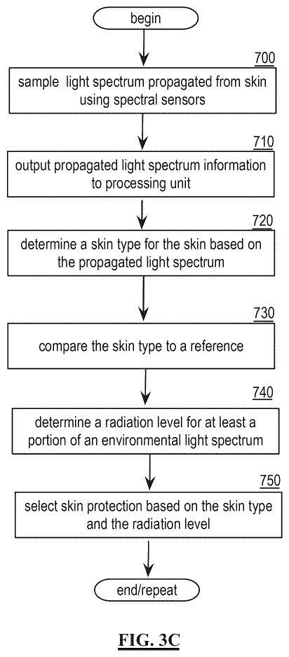

FIG. 3A is a flowchart illustrating an example method for determining a radiation exposure. The method begins at step 500, with one or more spectral sensors associated with a mobile device sampling a received light spectrum, where each of the one or more spectral sensors includes a plurality of interference filters overlaying one or more optical sensors, and each of the one or more spectrometers has a sensing range within a predetermined range of optical wavelengths, the sensing range for the plurality of interference filters together including a spectrum of wavelengths. The method continues at step 510 with the one or more spectral sensors outputting information representative of the received light spectrum to the one or more processing modules via one or more interfaces and based on the information representative of the received light spectrum and at step 520 determining, by the one or more processing modules, a radiation level for at least a portion of the received light spectrum. The method continues at step 530, with the one or more processing modules determining whether a predetermined threshold has been exceeded and when the predetermined threshold has been exceeded, notifying a user. When the predetermined threshold has not been exceeded, the method returns to step 500 for continued sampling.

In an example, the predetermined threshold can be a “snapshot” at the time of the sampling in step 500 and in a further example, the threshold can be based on a portion of the wavelengths in the received light spectrum. For example, the predetermined threshold can be based on the accumulated radiation according to wavelength, such that a portion of the light spectrum, such as, for example the portion of the light spectrum that includes the UVC wavelengths can have a predetermined threshold, past which the threshold is met.

In another example, the predetermined threshold can be a threshold of received radiation over a period of time. Accordingly, the accumulation of radiation at a given wavelength (or wavelengths) over a unit time can be used to predict when the radiation will exceed the predetermined threshold and once the predetermined of accumulated radiation is reached a threshold alert can be generated for transmission to a user. Moreover, the predetermined threshold for either a snapshot, accumulated radiation and rate of radiation accumulation can be based on a single wavelength, a plurality of wavelengths, or a full spectrum of wavelengths, with the breach of the predetermined threshold being used to generate a notification, alert or warning for any or all of the thresholding situations. The notification can be in the form of one or more of a display on a mobile device, an audible alert, an alert to a third party, such as health professional or a conservator.

In yet another example, the predetermined threshold can be based on a rate of radiation accumulation, such that the generation of a notification can be based on a rate of radiation accumulation for all or a portion of a spectrum of radiation. In a related example, the relationship between the rate of radiation accumulation and a predetermined threshold can be based on a training algorithm that is itself based on predetermined rules to predict when the predetermined threshold will be exceeded.

In an example, the predetermined threshold can based on a threshold reference, such as a reference database, where the reference database is stored locally or accessed via a network and where the database includes general radiation safety data or where the database is personalized to a particular classification of skin type or skin sensitivity. In another specific example, the threshold reference can be based on a prior classification of a particular user's skin using the spectral sensors of step 500. In yet another specific example, the classification of the user's skin can be determined based using a classification engine, such as a neural network and/or a cognitive computing engine.

In another example, the predetermined threshold (or thresholds) can be based on personal or general health data informed by crowdsourcing. For example, crowd sourced data can be used to inform one or more algorithms used to determine the predetermined threshold(s) for a particular user's skin classification. In an example, empirical data collected from a large number of skin types can be used to correlate safe radiation to each of the skin types, with that data being available to determine radiation thresholds for a particular user whose skin type is first classified using a spectrometer system, followed by analysis of the current radiation the user is being exposed to. In a specific example, the radiation threshold can be determined based on an accumulated radiation received in a given time period plus an expected radiation predicted to be accumulated at current or predicted radiation levels. In an example, predicted radiation can be based on a simple radiation over time calculus, or using more sophisticated mechanisms, such as historical patterns for the radiation relying on a large number of factors. Example factors can include, but are not limited to, time of day, season of the year, the activity being engaged in and mitigation of exposure due to the application of sun protection methods.

While being exposed to too much radiation in particular wavelength ranges can be unhealthy, nor spending enough time outside (or being otherwise exposed to certain light wavelengths) may itself be healthy. For example, lack of exposure to natural light can inhibit the production of vitamin D. Balancing optimal exposure to sunlight without overexposing a user to UV radiation can have health benefits. FIG. 3B is a flowchart illustrating an example method for determining an accumulated radiation exposure. The method begins at step 600, with one or more spectral sensors associated with the mobile device sampling a received light spectrum, where each of the one or more spectrometers includes a plurality of interference filters overlaying one or more optical sensors, and each of the one or more spectrometers has a sensing range within a predetermined range of optical wavelengths, the sensing range for the plurality of interference filters together including a spectrum of wavelengths. The method continues at step 610 with the one or more spectral sensors outputting information representative of the received light spectrum to the one or more processing modules via one or more interfaces and based on the information representative of the received light spectrum and at step 620 determining, by the one or more processing modules, an accumulated radiation level for at least a portion of the received light spectrum. The method continues at step 630, with the one or more processing modules determining whether a predetermined threshold has been met and when the predetermined threshold has been met, notifying a user. When the predetermined threshold has not been exceeded, the method continues at step 650, where a notification is generated notifying the user that a minimum radiation threshold has not been met. In an alternative example, the notification at step 650 includes an indication of the accumulated radiation level.

As discussed with reference to FIG. 3A, the predetermined threshold can be based on a variety of references, including personal and third-party sources. In a related example, the predetermined threshold can be a “snapshot” at the time of the sampling in step 600 and in a further example, the threshold can be based on a portion of the wavelengths in the received light spectrum. For example, the predetermined threshold can be based on the accumulated radiation according to wavelength, such that a portion of the light spectrum, such as, for example the portion of the light spectrum that includes the ultra-violet C (UVC) wavelengths (light between 200 nm and 280 nm) can have a predetermined threshold, past which the threshold is met.

Referring to the methods of both FIG. 3A and FIG. 3B, the predetermined threshold(s) can be based on a classification of skin type or skin sensitivity. In a specific example of implementation and operation, the classification is determined in an additional step prior to steps 500 and 600, respectively. In the example, the spectral sensors associated with the mobile device first sample a received light spectrum from a user's skin, the spectral response being used to classify the skin type of the user before sampling the received light spectrum. In an example, the classified skin type can be used to determine melanin levels of the skin and/or skin color to aid in determination of the predetermined threshold(s) for safe ultraviolet (UV) radiation instant and accumulated exposure level. In an example the skin classification for melanin can include a determination of eumelanin level and pheomelanin level, and in a related example, the ratio between eumelanin and pheomelanin is determined.

FIG. 3C is a flowchart illustrating an example method for classifying skin type for use in providing skin protection measures. The method begins at step 700, with one or more spectral sensors associated with a mobile device sampling a light spectrum propagated from skin or tissue, where each of the one or more spectrometers includes a plurality of interference filters overlaying one or more optical sensors, and each of the one or more spectrometers has a sensing range within a predetermined range of optical wavelengths, the sensing range for the plurality of interference filters together including a spectrum of wavelengths. The method continues at step 710 with the one or more spectral sensors outputting information representative of the propagated light spectrum to the one or more processing modules via one or more interfaces and based on the information representative of the propagated light spectrum at step 720 determining, by the one or more processing modules, a skin type for the skin. The method continues at step 730, with the one or more processing modules comparing the determined skin type to a reference mechanism and at step 740, a radiation level for at least a portion of an environmental light spectrum is determined. The environmental light spectrum can be determined in a manner consistent with FIGS. 3A and 3B, where the environmental light spectrum is a measure of the radiation the user is being exposed to. The method then continues at step 750 when, based on the comparing of the skin type to a reference mechanism and the determined environmental light spectrum, the one or more processing modules determine one or more skin protection measures for the skin. Skin protection measures can include a particular sun protection factor (SPF) sunscreen lotion and/or clothing for hair and/or skin, as dictated by the reference mechanism. The reference mechanism can be one or more of a database, a list, an expert system a classification mechanism, such as a trained neural network and can be locally stored and/or processed or retrieved from a cloud-based source.

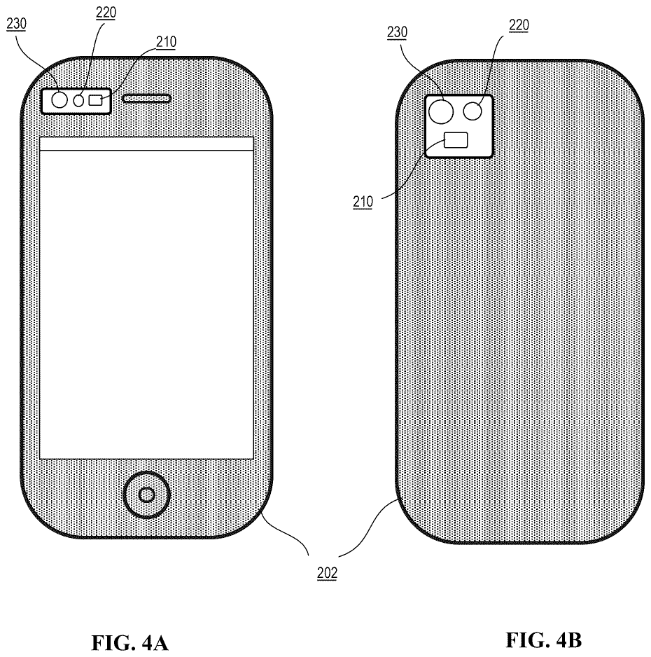

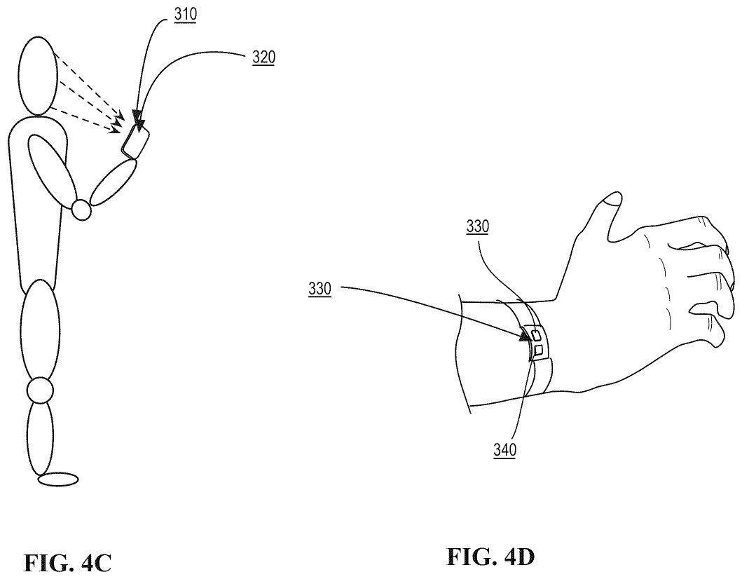

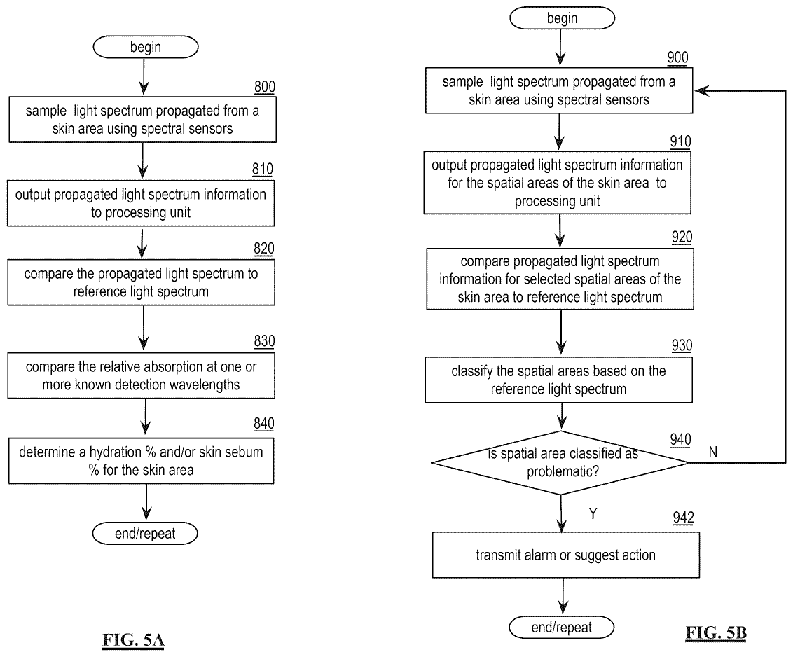

FIG. 4A illustrates a mobile device 202 with a forward-facing camera module incorporating an image sensor 230, a spectral sensor 210 and an illumination source 220. In an example, the forward-facing spectral sensor 210 can be used to collect a spectral response from a user's face or other body part. In a specific example, the forward-facing image sensor can be used to position the spectral sensor 210 on a particular body part, such as a section of skin, a skin aberration or other body parts, such as an eye, ear, lips or scalp. In a specific example, a mobile device display can provide targeting information for processing of the spectral response of the forward-facing spectral sensor. In another example, when a user is using the mobile device in “selfie mode” (i.e., to take self portraits), the forward-facing spectral sensor 210 can be used to automatically collect information about the user's facial features, either for use with the resultant selfie or for use later in another application. In a specific example, the spectral sensor 210 is adapted to function as an imaging device, so that an image of the body part can be provided either without the need for a separate image sensor or as an addition to a separate image sensor.

In another specific example of implementation and operation, a sensor system for imaging a body surface includes a plurality of optical sensors and a plurality of interference filters associated with the plurality of optical sensors. Each interference filter is configured to pass light in one of a plurality of wavelength ranges to one or more optical sensors of the plurality of optical sensors and each optical sensor of the plurality of optical sensors is associated with a spatial area of the body surface being imaged. In an example, a module of a processor (or multiple processors and/or modules), are adapted to produce a spectral response for one or more spatial areas of the body surface from the plurality of optical sensors, where the module (or modules) is adapted to determine one or more skin parameters for the spatial areas of the body surface. In an example, a display engine is included to output information representative of the one or more skin parameters for the spatial areas of the plurality of spatial areas of the body surface.

In an example, the skin parameters can include skin hydration and/or skin sebum (oiliness), which can be determined based on the spectral response for a spatial area of the skin/body surface. Skin hydration/skin sebum is associated with stratum corneum (SC) of skin, which is considered to be a barrier to water loss and is composed of the corneocytes and an intercellular lipid bilayer matrix. In a specific example of implementation, differential detection using three wavelengths 1720, 1750, and 1770 nm, corresponding to the lipid vibrational bands that lay “in between” the prominent water absorption bands can be used to approximate hydration levels and skin sebum in skin.

FIG. 4B illustrates a mobile device with a rear-facing camera module incorporating an image sensor, a spectral sensor and an illumination source. In an example, the rear-facing spectral sensor can be used to collect a spectral response from a user's skin or other body parts, such as the extremities, as well as spectral response from another user's face or other body parts. In another example, the rear-facing spectral sensor can be used to collect a spectral response to measure radiation levels in the environment, while the forward-facing spectral sensor is being used to measure a spectral response from a user's face or other body part. In a specific example, the mobile device display can be used to position the rear-facing spectral sensor on a particular body part and in another example, the mobile device display can provide targeting information for processing of the spectral response of the rear-facing spectral sensor. An illumination source (or sources) can be used to provide lighting for the image sensor and for collection of the spectral response from the spectral sensor when available.

FIG. 4C illustrates a mobile device with both a forward-facing spectral sensor and a back facing spectral sensor, allowing the collection of environmental radiation levels substantially concurrently with collection of spectral response from a user's skin or other body parts. In an example, when a user is using the mobile device in “selfie mode” (i.e. to take self-portraits), the forward-facing spectral sensor can be used to automatically collect information about the user's facial features, either for use with the resultant selfie or for use later in another application, while the rear-facing spectral sensor is available to collect a spectral response for incident light from the environment.

FIG. 4D illustrates a wrist mounted spectral sensor. In an example, a wearable device, such as a wrist mounted spectral sensor can incorporate one or more spectral sensors, allowing for the collection of environmental radiation levels and collection of a spectral response from a user's skin or other body parts. In a specific example, the wearable device may include one or more spectral sensors in contact or near contact with the skin, with an associated illumination source in contact or near contact with the skin located a predetermined distance from the one or more spectral sensors. In an example, the one or more spectral sensors can collect radiation reflected, scattered and transmitted by the illuminated skin and its components (such as layers, tissues, blood vessels, etc.). In an example, the wearable device can also include a spectral sensor and an optional illumination source facing away from the skin, allowing for relatively simultaneous collection of environmental radiation levels and spectral response from a user's skin. Other examples of wearable spectral sensors include sensors incorporated in smart-clothing and glasses/sunglasses.

Referring to FIGS. 2B and 4D, a camera module for a mobile device and/or a wearable spectral sensor can include an illumination source. Providing accurate spectral response form an object or environment requires a reliable reference spectrum of the illumination source used to illuminate a sample, such as skin, that is under study. In an example, a spectral sensor system can measure a reference spectrum by reflecting the light of the illumination source on a surface with a known spectral response immediately before measuring the spectral response of a sample. Illumination sources such as sunlight can be used with this method. However, more reliable spectral measurements can sometimes be obtained using illumination sources with known spectral emissions that are dedicated and controllable. Illuminations sources can provide wavelengths in the visible spectrum, as well as near-infrared (NIR), infrared (IR) and ultraviolet (UV) light wavelengths. Additional illumination sources include light emitting diode (LED) sources, such as wideband phosphor-coated LEDs. Illumination sources can include spectral filters for providing specific spectral output from the illumination sources. In an example, spectral filters can be used to reject certain wavelengths of light from the illumination sources or provide illumination in predetermined spectral bands.

In a specific example of implementation and operation, one or more illumination sources can be used to provide an illumination pattern, such as a striped pattern, one or more dots or other patterns that may be used in a spectral response. In an example, the illumination pattern allows for the spatial resolution of a surface being imaged along with spectral information. In another example, the illumination pattern enables three-dimensional (3D) depth spectroscopy imaging. In an example, the illumination patterns can be used to for detection of specific markers, such as health related skin markers. Related illumination sources can comprise advanced optics, such as dot pattern projectors and digital micro-mirror devices (DMDs). In an example a DMD is used to project patterned stripes on a surface being imaged.

In a specific example of implementation and operation, a device for measuring optical response from skin includes one or more illumination sources, where each of the one or more illumination sources is configured to provide light within a predetermined range of optical wavelengths. In an example, an illumination source can be configured to be adjustable to modify the illumination and the modified illumination can include a duty cycle for the illumination. The duty cycle is defined as a fraction of time in a time period during which the illumination source emits light, where a time period is the amount of time it takes for the illuminating source to complete an on-and-off cycle. Accordingly, modified illumination can include an increased duty cycle and a decreased duty cycle. Modified illumination can also be one of an increased current and a decreased current, where current is the power for an illumination source.

In a related example of implementation and operation, the one or more illumination sources can be optimized according to a sample, such as skin type or skin color under observation. Example skin types and/or skin colors include, but are not limited to phototypes on the Fitzpatrick scale and combinations of phototypes with other skin color factors, such as redness from blood. n an example of operation, a method for measuring spectrophotometric parameters of a sample of includes measuring spectrophotometric parameters of the sample using a first illumination “setting” (such as natural light, or using a default illumination) and then adjusting or modifying at least one illumination source of the one or more illumination sources based on the received light spectrum from one or more spectral sensors. In another example, modification of the illumination source(s) includes modulating at least one illumination source of the one or more illumination sources. In an example, the modulation can include illuminating according to a duty cycle, by sampling a first received light spectrum during a portion of time in the time period during when the at least one illumination source is not emitting light and sampling a second received light spectrum during a portion of time in the time period during when the at least one illumination source is emitting light. Modulation can allow the detection of background light and the measuring of its parameters, such as intensity, flickering and wavelength spectrum. This “background information” can then be used to correct the measurement of skin parameters by, for example, reducing background noise, etc.

In an example, the one or more illumination sources can be configured to irradiate light directly onto an area of skin. In an example, the device includes one or more spectrometers, wherein each of the one or more spectrometers includes a plurality of interference filters overlaying one or more optical sensors and each of the one or more spectrometers has a sensing range within a predetermined range of optical wavelengths. The one or more spectrometers are configured to capture light emitted from the skin and are positioned a predetermined distance from at least one illumination source. The one or more spectrometers are configured to output information representative of a spectral response to one or more modules of a processing device that is itself adapted to produce a spectral response for at least a portion of one spectrometer of the one or more spectrometers and is further adapted to determine one or more skin parameters for the skin.

In a specific example of implementation and operation, a device for measuring optical response from skin includes one or more illumination sources, where each of the illumination sources is configured to provide light within a predetermined range of optical wavelengths, and the illumination sources are configured to irradiate light directly onto skin. In an example at least one of illumination sources is adapted for modulation. In an example, the device includes one or more spectrometers, each of the spectrometers including a plurality of interference filters overlaying one or more optical sensors. In an example, each of the spectrometers has a sensing range within a predetermined range of optical wavelengths and is configured to capture light emitted from the skin, where each of the spectrometers is positioned a predetermined distance from at least one illumination source of the one or more illumination sources.

In an example, the device includes a processor with a first module configured to receive an output from the spectrometers and a second module configured to determine one or more skin parameters based on the output from the one or more spectrometers. In a specific example, the modulation of the illuminating device(s) includes modulating the illumination according to a duty cycle, where the duty cycle is a fraction of time in a time period during which the one or more properties of the illuminating device is being varied. In a specific example the duty factor for the illumination can be scaled to a maximum of one or to a maximum of 100% illumination. In an example, the properties can be one or more of intensity, wavelength, etc. and the modulation can be in the form of a sine wave, a modified sine wave, a square wave or any other practical waveform.

In a specific example, a processor is further configured to receive the output from the one or more spectrometers during both a time period when one or more properties is being varied and during a time period when the one or more properties is not being varied.

FIG. 5A is a flowchart illustrating an example method for determining skin parameters. The method begins at step 800, where a light spectrum propagated from an area of skin is sampled using spectral sensors and continues at step 810 with the sampled light spectrum being output to a processing device. In an example, an illumination source of predetermined wavelengths is used to illuminate the skin sample and in another example the illumination source is natural light. In another example, the illumination source wavelengths and intensity are determined prior to sampling the propagated light spectrum and then used to compensate for nonideal illumination of the skin area. The skin area can be all or a portion of the spatial area of a scene or object being imaged with a mobile device image sensor. The method continues at step 820, where the propagated light spectrum is compared to a reference light spectrum. In an example, the reference light spectrum is predetermined based on data collected previously on the area of skin. In another example, the reference light spectrum is based on empirical data and crowd-sourced data.

The method continues at step 830, with the relative absorption at one or more detection wavelengths being determined based on the comparison of the propagated light spectrum with the reference light spectrum. In an example, the detection wavelengths are wavelengths that correlate to a particular skin and/or tissue parameter, such as skin hydration and/or skin sebum. The method continues at step 840, with the processing device determining a skin parameter percentage (%) such as a hydration percentage (%) and/or skin sebum percentage (%) based on the relative absorption at the detection wavelengths.

In an optional step the determined skin parameter percentage (%) can be output for display on a mobile device, such as a smart mobile phone, with the mobile device displaying the percentages as level indicators for a spatial area of a scene or object imaged by an image sensor. For example, a large skin area might display a level indicator for one or more skin parameters in each of a plurality of spatial areas of an image of a scene or object. In another example, one or more spatial areas of an image of a scene or object can include a potential skin aberration, with the display providing comparative indicators for one or more skin parameters for the potential skin aberration and unaffected skin. In the example, the comparative indicators can provide diagnostic information relative to the potential skin aberration.

FIG. 5B is a flowchart illustrating an example method for detecting and classifying skin aberrations. The method begins at step 900, where a light spectrum propagated from an area of skin is sampled using spectral sensors and continues at step 910 with the sampled light spectrum for one or more spatial areas of the area of skin being output to a processing device. The method continues at step 920, where the propagated light spectrum for the one or more spatial areas of the area of skin are compared to reference light spectra. In an example, the reference light spectra are based on spectra collected previously on the spatial areas. The method continues at step 930, with the spatial areas being classified based on the reference light spectra. In an example, the classification is further based on changes to one or more of spatial areas as compared to previously collected spectra. In another example, the classification is based on comparison to known and/or predetermined spectra, where the known and/or predetermined spectra are associated with one or more skin conditions and/or diseases. The known and/or predetermined spectra can be stored locally or collected from an outside database. In a specific example the classification is determined using a trained neural network and/or using a cognitive computing engine, either of which can be local to the spectral sensor/mobile device or networked to the mobile device.

The method continues at step 940, with the processor determining whether the spatial area classification indicates a disease, skin condition or other aberration and when the classification indicates a disease, skin condition or other aberration, at step 950 the processor generates an alarm and/or suggests a proposed action for the disease, skin condition or other aberration. In an example, the classification can include an indication of disease or skin condition for use by the processor to determine whether to generate and transmit an alarm or suggest an action. If the spatial area classification does not indicate a problem the method reverts to step 900. Example skin aberrations can include healthy and malignant moles, skin melanomas, psoriasis, basal skin carcinoma and virtually any other skin-based malady.

In another example of implementation and operation, a first propagated light spectrum is used as reference light spectrum and a second propagated light spectrum is compared to the first propagated light spectrum for classification of one or more spatial areas of skin. For example, the first propagated light spectrum can be from a skin area with known healthy skin, with the second propagated light spectrum being from a skin area with one or more potential skin aberrations. In another example, the first propagated light spectrum can be from an earlier observation of a same skin area. In yet another related example, the first propagated light spectrum can be from a skin area with known healthy skin, which is then used to calibrate the spectrophotometric parameters for a plurality of subsequent parameter measurements. And, in yet another example, the first propagated light spectrum can be from a skin area with a skin aberration, such as a wound, diseased or infected skin, with the second propagated light spectrum being used to determine a change to the skin aberration, where the change can be used to provide, for example, an indication of healing, a worsening of the aberration (such as an infection, etc.)

In a related example of operation, the classification can include a first propagated light spectrum used as reference light spectrum and a second propagated light spectrum, where the first propagated light spectrum is from a known healthy area of skin and the second propagated light spectrum is used to determine changes to specific skin parameters, such as skin color or other skin spectrum differences and used to classify a skin aberration or other skin feature. For example, the identification of a problematic skin mole or potential skin melanoma might be aided at least in part on differences between a known healthy skin measurement and a potentially problematic skin area.

In an example, either one of the classification or suggested action can be determined at least partially based on one or more of the idiosyncratic skin type of a user, genetic information related to the user, hair color, eye color and can be determined at least partially on changes over to time or on a single sample. In an example, collected classification information can be shared with a crowd-sourced database for use in training a neural network and/or cognitive computing engine.

In an example, the method of FIG. 5B can initiated on an ad hoc basis by a user or executed automatically as a skin area is imaged. In a related example, the method can be implemented as a background operation, or it can be triggered when a predetermined period of time has elapsed. In another example, the body surface includes at least a portion of a user's eye and wherein the processing device is adapted to determine a near-infrared (NIR) spectrum of the eye. In an example, the NIR spectrum can be used to assist in biometric analysis of the user, in addition to normal visible information obtained with an iris reader.

In a specific example of implementation and operation related to FIGS. 5A and 5B, spectral sensors can be used in combination with other diagnostic mechanisms to determine health parameters. In an example, a contact lens (or any other device configured to maintain physical contact) incorporating a glucose-detecting passive sensor such as a hydrogel can be worn, where the passive sensor is adapted to be spectroscopically chromophoric to detected glucose. In an example, a user can assess the glucose level by taking a spectral image of the eye. In an example, the assessed glucose level can then be correlated to a user's glucose levels. In an example, the spectral image can be provided using a mobile device camera and, in another example, an eye-facing camera may be installed in smart glasses for manual or semi-continuous monitoring of glucose levels. In an example, other health parameters can be assessed, including lactate levels.

In a specific example of implementation and operation, a passive glucose sensor, such as the glucose sensor described above can include a non-responsive sensor or section adjacent to a responsive sensor or section of the contact lens, such that a differential or ratio-metric measurement can be performed to determine issues associated with background light. In another example, a controlled active light source is incorporated in the diagnostic mechanism. In a related example, infrared light is used instead of visible light so that a user's sight is not affected by the measurement. In yet another example, an eye-facing spectral camera can be used in smart glasses or another wearable device, to measure ophthalmological issues with the eye. Examples include using the spectroscopic data to locate and/or measure blood vessels in the eye.

In an example of implementation, an optional illumination source (or sources) can be included to provide lighting for the image sensor and for collection of the spectral response from the spectral sensor when available. In a further example, a spectral sensor can provide spatial and spectral information for a scene or object being imaged by the image sensor.

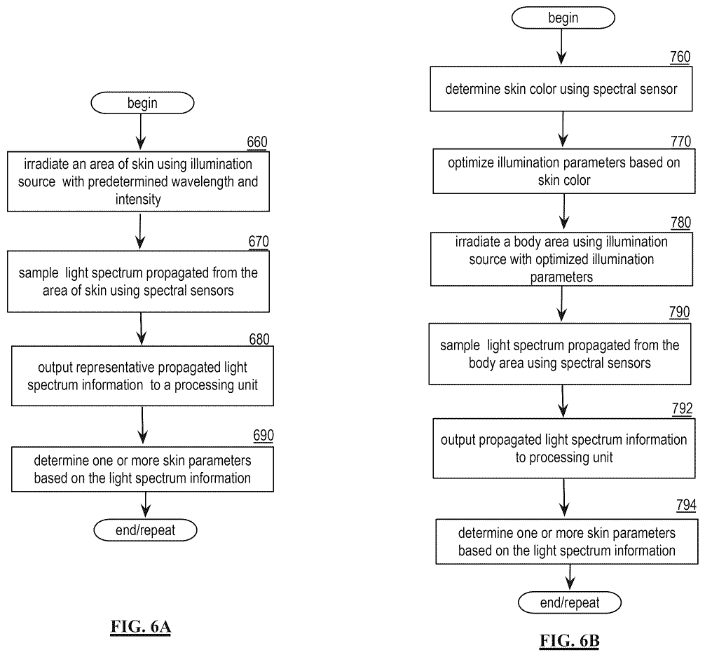

FIG. 6A is a flowchart illustrating an example method for determining skin parameters using a spectral sensor. The method begins at step 660, with an area of skin being irradiated by one or more illumination sources, where each of the one or more illumination sources is configured to provide light within a predetermined range of optical wavelengths and are configured to irradiate light directly onto the area of skin. In an example, the illumination sources are additionally configured to provide light across the predetermined range of optical wavelengths at a predetermined intensity. The method continues at step 670 with one or more spectral sensors sampling a received light spectrum from the area of skin, where each of the one or more spectrometers includes a plurality of interference filters overlaying one or more optical sensors and each of the one or more spectrometers has a sensing range within a predetermined range of optical wavelengths. In an example, each of the one or more spectrometers is configured to capture light emitted from the area of skin and each of the one or more spectrometers is positioned a predetermined distance from at least one illumination source.

At step 680 the one or more spectrometers output information representing the response of the one or more spectrometers to one or more modules of a processing device and at step 690 the processing device determines one or more skin parameters for at least a portion of the area of skin. In an example, the one or more skin parameters can be determined at least partially based on a comparison of the response from the one or more spectrometers with a reference response, where the reference response is one or more of a response database, a comparison with an earlier stored response and a classification engine (such as a neural network or cognitive computing engine).

In another example, the skin parameters can be determined based on a compound classification using a matrix of illumination intensities and light wavelengths and in another example, the matrix of illumination intensities and light wavelengths can be used to train a neural network for classifying a response determination of one or more skin parameters. In another example the neural network can be trained using a mean testing scheme over a period of time.

In another specific example of implementation and operation, a device for measuring optical response from skin includes one or more illumination sources, where each of the one or more illumination sources is configured to provide light within a predetermined range of optical wavelengths and the one or more illumination sources is further configured to irradiate light directly onto skin. In an example, the device includes one or more spectrometers configured to capture a spatial image of a scene, where each of the one or more spectrometers includes a plurality of interference filters overlaying one or more optical sensors. In an example, each of the one or more spectrometers has a sensing range within a predetermined range of optical wavelengths and is configured to capture light emitted from the skin, further wherein each of the one or more spectrometers is positioned a predetermined distance from at least one illumination source of the one or more illumination sources. In an example, the device includes a first module of a processor that is configured to receive an output from the one or more spectrometers that includes an image of the scene and a received light spectrum, a second module of the processor is configured to determine one or more skin parameters based on the output from the one or more spectrometers, where the second module further configured to store the one or more skin parameters in memory. In an example, the device includes a third module of the processor configured to compare the one or more skin parameters with one or more references. In an example, the references can include an earlier image and/or received light spectrum. In another example, the references include a compilation of skin parameters collected from 3^{rd} party sources.

In a specific example of implementation and operation, at least one of the one or more illumination sources is adapted to provide variable power, and in another example, the one or more illumination sources is adapted to provide variable intensity.

FIG. 6B is a flowchart illustrating another example method for determining skin parameters using a spectral sensor. The method begins at step 760, with the spectral sensor being used to determine the skin color of an area of skin using, for example, the method illustrated in in steps 800-820 of FIG. 5A. The method continues at step 770, with the illumination parameters for one or more illumination sources being optimized based on the determined skin color. Illumination parameters can include increasing or decreasing the duty cycle and/or current of the one or more illumination sources. In a representative example the duty cycle and/or current of a light emitting diode (LED) illumination source can be increased for dark skin color and decreased for pale or light skin color, thereby increasing signal to noise ratio of a spectral response where possible. The method continues at step 780 with the area of skin being irradiated by one or more illumination sources and continues at step 790 with one or more spectral sensors sampling a received light spectrum from the area of skin, where each of the one or more spectrometers includes a plurality of interference filters overlaying one or more optical sensors and each of the one or more spectrometers has a sensing range within a predetermined range of optical wavelengths. In an example, each of the one or more spectrometers is configured to capture light emitted from the area of skin and each of the one or more spectrometers is positioned a predetermined distance from at least one illumination source.

At step 792 the one or more spectrometers output information representing the response of the one or more spectrometers to one or more modules of a processing device and at step 794 the processing device determines one or more skin parameters for at least a portion of the area of skin based on the output information.

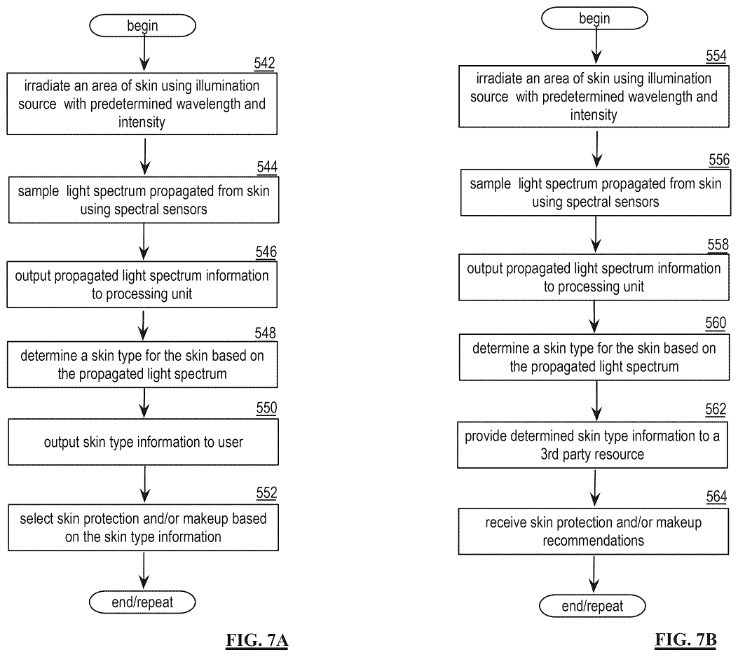

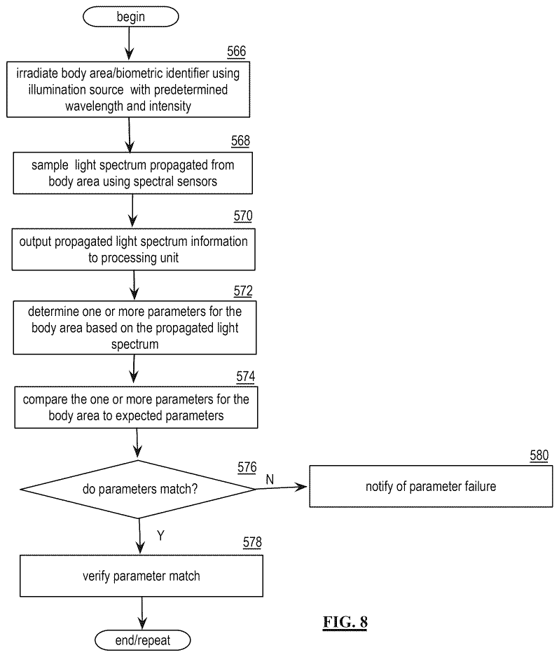

FIG. 7A is a flowchart illustrating an example method for classifying skin type for use in providing skin treatment. The method begins at step 542, with an area of skin being irradiated by one or more illumination sources, where each of the one or more illumination sources is configured to provide light within a predetermined range of optical wavelengths and are configured to irradiate light directly onto the area of skin. The method continues at step 544, with one or more spectral sensors associated with a mobile device sampling a propagated light spectrum from skin or tissue, where each of the one or more spectrometers includes a plurality of interference filters overlaying one or more optical sensors, and each of the one or more spectrometers has a sensing range within a predetermined range of optical wavelengths, the sensing range for the plurality of interference filters together including a spectrum of wavelengths. The method continues at step 546 with the one or more spectral sensors outputting information representative of the propagated light spectrum to the one or more processing modules via one or more interfaces and based on the information representative of the propagated light spectrum at step 548 determining, by the one or more processing modules, a skin type for the skin. In an example, the skin type can be a measure of the melanin in the skin area, skin color, etc. as discussed in further detail below.

The method continues at step 550, with the one or more processing modules outputting the skin type information to a user. In an example, the skin type information can be displayed on an associated mobile device and in a further example, can be in the form of a reference identifier, such as a code or a simple identifier associated with a number or other identifier reference for use by the user. For example, the skin type information could be displayed as a basic skin tone with an alphanumeric indicating a gradation within the basic skin tone. Basic skin tone can, for example, be identified as one of “fair”, “light”, “medium” or deep, with number from 1-5 indicating the gradations. Skin type information can also include skin undertones within a basic skin type, such as cool, warm and neutral. Other options for skin type information display include bar code, or other code-based representation that can be used to match the skin type information with a reference source. In a related example, skin type information can include additional skin factors, such as hydration level, dryness, roughness, oiliness, and flakiness, along with combinations thereof.

The method then continues at step 552 when, based on the skin type information, a user can select skin treatment. Skin protection measures, makeup, moisturizers, etc. for the skin. In an example, skin treatment can include one or more of a type, brand and dose of make-up, a particular sun protection factor (SPF) sunscreen lotion and/or clothing for hair and/or skin. When the method of 7A is used on skin to which makeup and/or other treatment has already applied, skin type information can also be used to make changes to the makeup and/or other treatment to correct the makeup application. In an example, skin type information can be used to provide a recommended skin treatment and after the skin treatment is applied, a second scan or analysis can be used to assess the effectiveness of the applied skin treatment and/or provide corrective actions.

In a specific example, various skin parameters and levels, such as skin type, skin color, hydration, oiliness, and melanin concentration can be determined in a plurality of skin “zones”. The zone-based skin parameters can be used to adjust and/or optimize moisturizer, sunscreen, and makeup for each different skin area. In a related example, skin parameters such as skin color, hydration level, melanin concentration can be used to identify a healthy and unhealthy skin zones, where an unhealthy skin zone can have infected or healing skin. The skin parameters for one or more healthy zones can be used as a reference to determine, for example, the severity of an infection and/or to monitor a skin healing process. In another example the unhealthy skin zone can include a skin zone with a skin mole or suspected melanoma. In the example, the skin parameters for one or more healthy zones can be used as reference to classify the skin moles and/or identify the melanoma.

FIG. 7B is a flowchart illustrating another example method for classifying skin type for use in providing skin treatment. The method begins at step 554, with an area of skin being irradiated by one or more illumination sources, where each of the one or more illumination sources is configured to provide light within a predetermined range of optical wavelengths and are configured to irradiate light directly onto the area of skin. The method continues at step 556, with one or more spectral sensors associated with a mobile device sampling a propagated light spectrum from skin or tissue, where each of the one or more spectrometers includes a plurality of interference filters overlaying one or more optical sensors, and each of the one or more spectrometers has a sensing range within a predetermined range of optical wavelengths, the sensing range for the plurality of interference filters together including a spectrum of wavelengths. The method continues at step 558 with the one or more spectral sensors outputting information representative of the propagated light spectrum to the one or more processing modules via one or more interfaces and based on the information representative of the propagated light spectrum at step 560 determining, by the one or more processing modules, information representative of skin type for the skin.

The method continues at step 562, with the one or more processing modules outputting the skin type information for use by a 3^{rd} party. In an example, the skin type can be provided using a communication mechanism associated with a mobile device automatically or in response to a prompt to a user. In a specific example, a vendor/advertiser can provide a prompt to a user's mobile device prompting the user to scan their skin using the spectrometer on the mobile device and, when the user responds by scanning their skin the vendor/advertiser can then use the skin type to determine an appropriate skin treatment for the user. In a related example, the skin type information can be provided to the 3^{rd} party using direct communication, such as by transmitting/relaying the skin type information in the form it is received by a user. In another example, the skin type information can be provided as a bar code, a Quick Response (QR) code or other form that can be provided to the 3^{rd} party using a user's mobile device.

The method then continues at step 564 with receipt of a recommendation from the 3rd party for skin treatment. In an example, the user can be an individual consumer interacting with the 3^{rd} party over a cloud-based network, such as the internet, using their own mobile device. In another example, the user can be a service provider, such as a cosmetologist or a health care provider interacting with the 3^{rd} party over a local and/or cloud-based network.

Biometrics authentication (sometimes called realistic authentication) can be used as a form of identification and access control. While biometric identifiers are considered to be distinctive, measurable characteristics of a person, the measurement and analysis is not always perfect. Moreover, while biometric authentication is intended to improve authentication accuracy, it is also desirable that the authentication does not add unnecessary burden to an authentication process. Example biometric identifiers include, but are not limited to fingerprints, palm veins, face recognition, palmprint, hand geometry, iris recognition, and retina, each of which involve presenting a body part for biometric measurement. In practice, a biometric authentication system can require two or more additional identifiers in order to improve accuracy, however adding such additional identifiers can add extra burden to a user.

FIG. 8 is a flowchart illustrating an example method for using body area parameters from spectral sensing for biometric analysis. The method begins at step 566, with a biometric authentication system irradiating a body area/biometric identifier, such as one or more fingerprints, palm veins, facial area, palmprint, hand geometry, iris recognition, and retina by one or more illumination sources, where each of the one or more illumination sources is configured to provide light within a predetermined range of optical wavelengths and are configured to irradiate light directly onto a body area (biometric identifier) being used for biometric identification. The method continues at step 568, with one or more spectral sensors sampling a propagated light spectrum from the biometric identifier, where each of the one or more spectrometers includes a plurality of interference filters overlaying one or more optical sensors, and each of the one or more spectrometers has a sensing range within a predetermined range of optical wavelengths, the sensing range for the plurality of interference filters together including a spectrum of wavelengths.

The method continues at step 570 with the one or more spectral sensors outputting information representative of a propagated light spectrum to the one or more processing modules via one or more interfaces and based on the information representative of the propagated light spectrum at step 572 determining, by the one or more processing modules, information representative of one or more parameters for the biometric identifier based on the propagated light spectrum. In an example, the one or more parameters can be one or more of melanin concentration of skin, skin color, blood flow patterns, comparative wavelengths of tissue areas, such as blood vessels compared to surrounding tissue, etc. Additional parameters can include temperature, as determined by absorption/reflection in infrared (IR) wavelengths, blood flow (for example whether blood is flowing and/or a rate of blood flow) and the presence or absence of skin impurities and/or aberrations. In another example, when the biometric identifier is a retina or iris, absorption/reflection in near-infrared (NIR) wavelengths can provide additional identifying parameters for the retina or iris being authenticated.

In a specific example of implementation, the spectral sensors can be incorporated in smart glasses that are coupled to a mobile device, so that the output of the spectral sensors can be collected by the smart glasses and used to authenticate the mobile device. In another example, the mobile phone can be used to authenticate the wearer of the coupled smart glasses. In a related example, the output of the spectral sensors can be collected by the smart glasses and used to authenticate other devices, such as commercial vehicles (such as trains, trucks and planes, for example) and/or for authentication of safety devices in order to prevent unauthorized use. In the example, the authentication can be manually activated by a user and/or a third party and in another example, authentication could occur transparently, so that a user or users need not be burdened by the authentication process.

The method continues at step 574, with the biometric authentication system comparing the information representative of one or more parameters to “expected” parameters for the person being authenticated and determining at step 576 whether the parameters match the expected parameters. At step 578 the parameters match within a predetermined threshold of accuracy, using the positive match as a second authentication factor for the biometric identifier. The biometric authentication system can use this second authentication factor to augment the accuracy of the system without additional authentication requirements. When the body area parameters do not meet a match threshold the biometric authentication system can use this second authentication factor as an indication of non-authentication. As will be apparent to one skilled in the art, when a biometric identifier is being presented for authentication, the method of FIG. 8 can provide a second authentication while adding little additional burden to the authentication process. In an example, the parameters of the biometric identifier can be collected in a manner transparent to the authentication subject.

In specific example, the spectral sensors can be configured to provide spatial information along with spectral information, where the spectral information can be used to determine/confirm that the biometric identifier, such as the iris of an eye, is from an actual face/person. In an example, the spectral sensors can provide additional spectral information in addition to spatial information of a biometric identifier that can be used for authentication purposes.

Referring to FIGS. 2A and 2B, image sensors can be provisioned with spectral sensors in a camera module for a mobile device and in a specific related example of implementation and operation, an imaging system includes an image sensor including a set of associated optical sensors. In an example, the optical sensors are red, green, blue (RGB) color channel sensors configured to capture information from a scene or image in the visible spectrum. In an alternative example, the image sensor is also a spectral imager. In an example, a plurality of interference filters is associated with another (second) set of optical sensors, where each interference filter of the plurality of interference filters is configured to pass light in one of a plurality of wavelength ranges to one or more optical sensors of the second plurality of optical sensors. In an example, each optical sensor of the second plurality of optical sensors is associated with a spatial area of the image and the plurality of wavelength ranges for the plurality of filters includes wavelengths extending beyond the range of the image sensor. In an example, the wavelength ranges extending beyond the range of the RGB sensors include one or more of IR, MIR, NIR, Deep UV and UV wavelengths.

In an example, the image sensor output, when added to the spectral sensor information in the extended wavelength ranges, can be used to provide additional information for determination of the spectral information. For example, the additional information can be used to provide precision to the determination of skin color and other use cases described herein. FIG. 9 is a flowchart illustrating an example method for using the combined output from an image sensor and a spectral sensor. The method begins at step 842, by irradiating an area of skin or other body area using one or more illumination sources, the body area with light, where each of the one or more illumination sources is configured to provide light within a predetermined range of optical wavelengths, and where the one or more illumination sources is further configured to irradiate light directly onto the skin/body area. The method continues at step 844 by generating an image of at least a portion of the skin/body area, where the generating is based on an output from an image sensor. The method continues at step 846 by sampling a received light spectrum from one or more spectral sensors, wherein each of the one or more spectrometers includes a plurality of interference filters overlaying one or more optical sensors, wherein each of the one or more spectrometers has a sensing range within a predetermined range of optical wavelengths and is configured to capture light emitted from the body area, wherein each of the one or more spectrometers is positioned a predetermined distance from at least one illumination source of the one or more illumination sources. The method then continues at step 848 by outputting information representative of the propagated light spectrum information to a processing unit and modifying, at step 850 the received light spectrum information from the one or more spectral sensors based on the image generated by the image sensor. At step 852, one or more skin and/or body parameters are determined based on the modified received light spectrum information.

In a specific example of implementation, the body area includes one or more areas that include skin and the one or more spectrometers are adapted to capture the received light spectrum from at least one of the one or more areas that include skin. In another example, the image sensor includes red, green, blue (RGB) color channel sensors and the plurality of wavelength ranges for the plurality of filters includes wavelengths extending beyond the range of the image sensor. In an example, the wavelength ranges extending beyond the range of the RGB sensors include one or more of IR, MIR, NIR, Deep UV and UV wavelengths.

In another example, the determining of the one or more skin and/or body parameters are determined based on the modified received light spectrum information includes classifying at least a portion of the skin or body area based on the modified received light spectrum.

A specific embodiment includes using spectral measurements to determine pressure exerted on skin or other tissue. For example, when pressure is applied to skin, blood is pushed away from the skin surface and it will no longer be detectable in the outer layers of the skin. Different wavelengths emitted by an illumination source penetrate to different skin depths; for example, longer wavelengths penetrate deeper into the skin and shorter wavelengths only reach the outer layers of skin. Accordingly, shorter wavelengths, by not penetrating, will not exhibit an interaction with blood when pressure is applied to the skin. The absence of such an interaction will be exhibited as a change in the spectrum detected by a spectral sensor.

These changes in spectrum can be used to measure an amount of pressure applied to the skin. In a related example, the skin pressure information derived from changes in a received spectrum can be used to correct sensory data that may be sensitive to pressure, such as data obtained from heart rate sensors, blood oxygen saturation (SpO₂) sensors, electrocardiogram (ECG) electrodes, galvanic skin sensors, skin temperature sensors, etc. In an example, a correction can include compensation for pressure exerted by a sensor on skin and/or compensate for the depth of blood under the skin surface. In a related example, a measurement of the depth of blood under the skin surface can be used to correlate skin temperature and body core temperature.

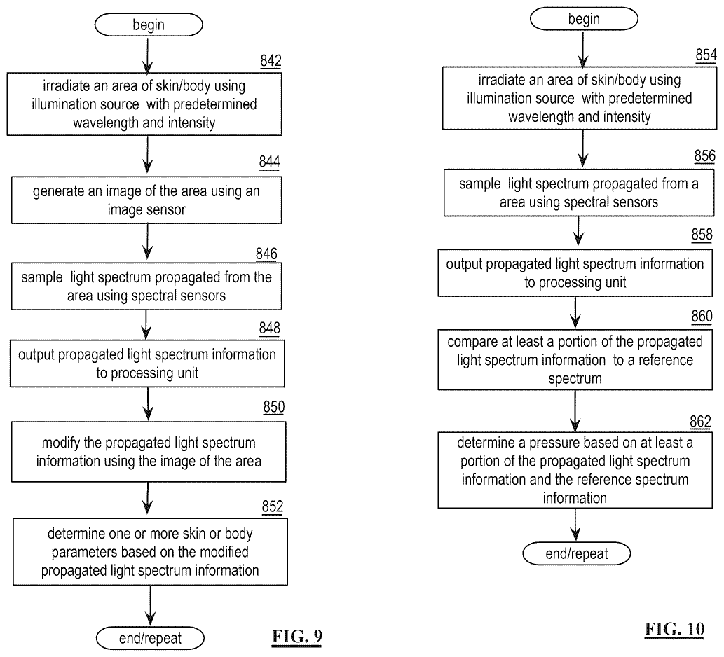

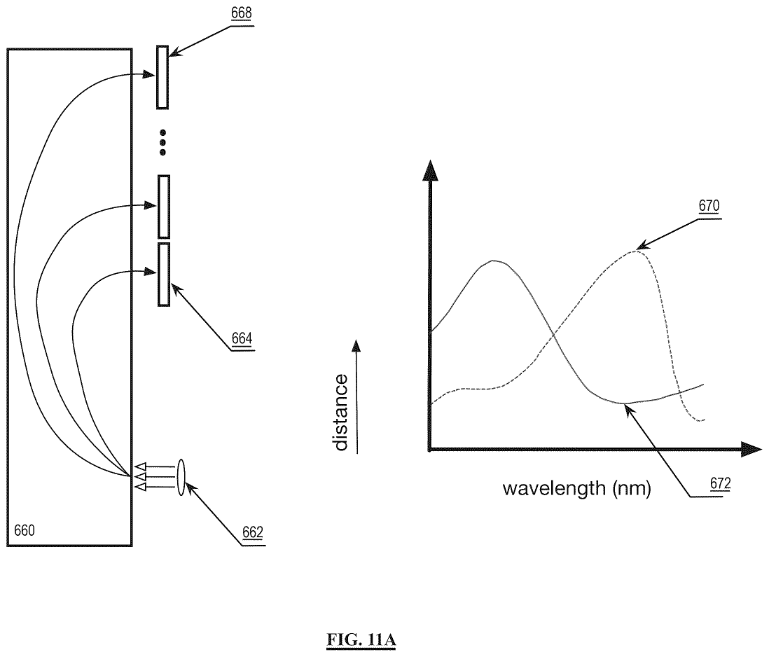

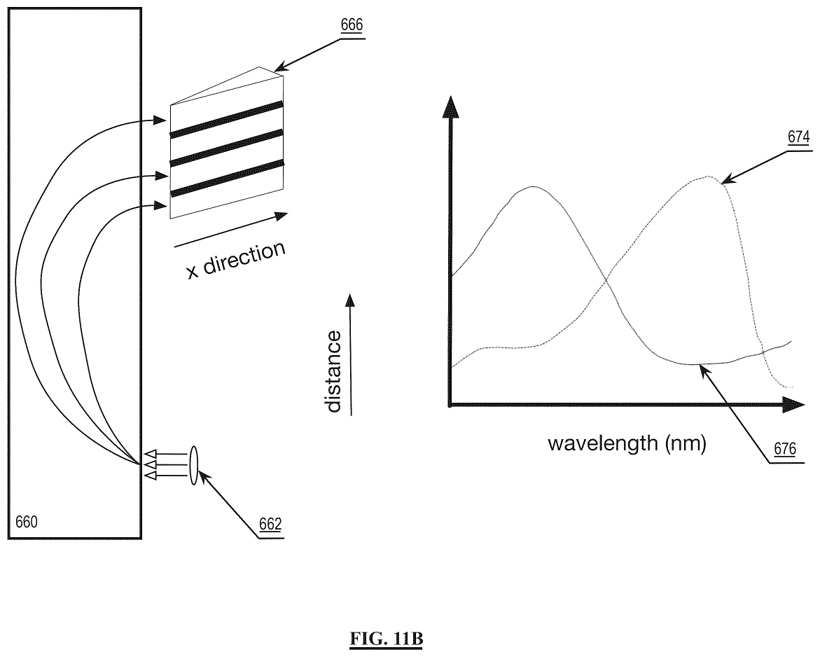

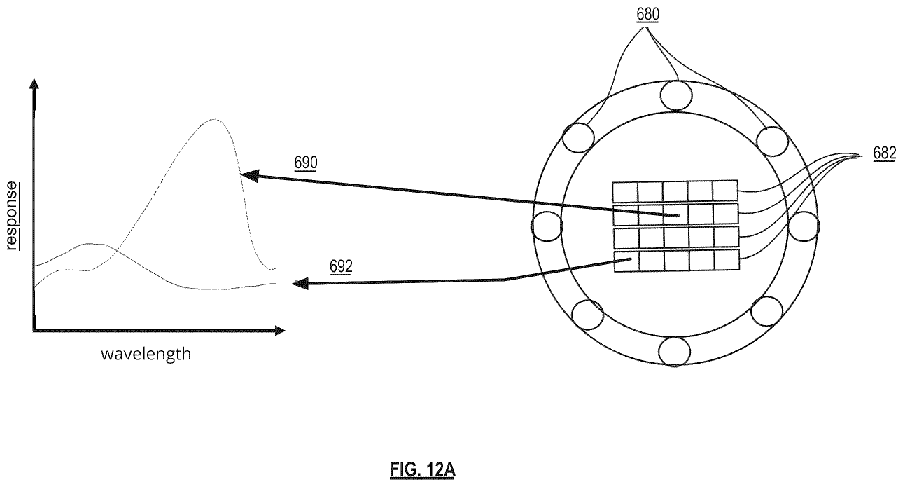

FIG. 10 is a flowchart illustrating an example method for determining applied pressure using a spectral sensor. The method begins at step 854, by irradiating an area of skin, with light using one or more illumination sources, where each of the one or more illumination sources is configured to provide light within a predetermined range of optical wavelengths, and where the one or more illumination sources is further configured to irradiate light directly onto the skin/body area. The method continues at step 856 by sampling a received light spectrum from one or more spectral sensors, where each of the one or more spectrometers includes a plurality of interference filters overlaying one or more optical sensors. In an example, each of the one or more spectrometers has a sensing range within a predetermined range of optical wavelengths and is configured to capture light emitted from the skin area. In an example, each of the one or more spectrometers is positioned a predetermined distance from at least one illumination source of the one or more illumination sources. The method then continues at step 858 by outputting information representative of the propagated light spectrum information to a processing unit and at step 860 comparing the received light spectrum information to a reference spectrum.

In an example, the comparison can be based on a portion of the and the comparison can be based on particular wavelengths of the received light spectrum. For example, the comparison might be based on only the portion of the received light spectrum required to determine blood in the skin. At step 862, the comparison of the received light spectrum and the reference spectrum can be used determine a pressure on the skin. For example, the comparison can show that in shorter wavelength ranges of the received light spectrum blood is not detected as compared to the reference spectrum, indicating a relative pressure increase on the skin observed in the received light spectrum.

In an example, the reference spectrum can be a previously received light spectrum, with the difference indicating a change in pressure. In another example, the reference spectrum can be a database or list that correlates a received light spectrum to pressure range. In yet another example, the determined pressure can be provided along with data collected from another sensor to enable analysis using the other sensor.

FIG. 11A provides an illustration of a spectral sensing system incorporating multiple spectral sensors 664-668, each located a different predetermined distance from an illumination source 662. In the example, a spectral sensing system includes multiple spectral sensors 664-668 configured adjacent to each other. In the example one or more illumination sources 662 can be configured to illuminate a sample 660, such as skin, tissue, liquid, etc., with light propagated from the sample 660 collected at the multiple spectral sensors 664-668. The responses of the spectral sensors are defined by their relative distance to the illumination source, for example, longer wavelengths penetrate deeper into the skin and shorter wavelengths only reach the outer layers of skin. In the example, photons travelling deeper into skin or other tissue would provide a spectral response primarily on the spectral sensor (such as spectral sensor 668) farthest from the illumination source 662 and photons travelling at a shallower angle into skin, would appear on the spectral sensors (such as spectral sensor 664 et. sec.) closest to the illumination source. In an example, the spectral response across the spectral sensors 664-668 can be used to provide substantially simultaneous analysis at different depths in the skin or tissue sample. In the example, the spectral analysis can then use, for example, a differential comparison of the spectral sensor responses to better understand the skin or tissue sample. In another example, the successive spectral sensors can be positioned at higher or lower distances relative to the substrate that the spectral sensors are mounted on.

FIG. 11B provides another illustration of a spectral sensing system incorporating multiple spectrometers (embodied together as sensor wedge 666) located at different distances from an illumination source 662. In the example, a spectral sensing system comprises multiple spectral sensors configured adjacent to each other, with each successive spectrometer being at little higher or lower distance relative to a substrate that the spectrometers are mounted on. In the example an illumination source 662 (or illumination sources) is configured to illuminate a sample 660, such as skin, liquid, etc., with light propagated from the sample 660 collected at multiple spectral sensors of sensor wedge 666, each of which receives propagated light at a different distance relative to the sample 660. In a specific example, multiple spectral sensors are configured to form a sensor wedge 666, where each spectral sensor is a different distance relative to the sample 660. In a related example, when the illumination source 662 is natural light, such as direct or filtered sun light, the illuminating can be from a specific angle relative to the sample 660 and in another example the illuminating can be from a plurality of diffuse angles and locations. In another related example, when the illumination source 662 is artificial light, such as one or more light emitting diodes (LEDs), the illuminating can also be from an angle relative to the sample 660 and in another example the illuminating can be from a plurality of diverse angles surrounding the sample 660. In alternate example, the multiple spectral sensors of spectral wedge 666 can be at a substantially same level and configured so that one or more spectral sensors are level with each other and tilted and/or rotated relative to the sample 660 or the illumination source(s) 662. In another alternative example, the multiple spectral sensors can be configured in a wedge 666 such that each spectral sensor is at a higher or lower level relative to the sample 660 and tilted and/or rotated relative to the sample 660 or the illumination source(s) 662.

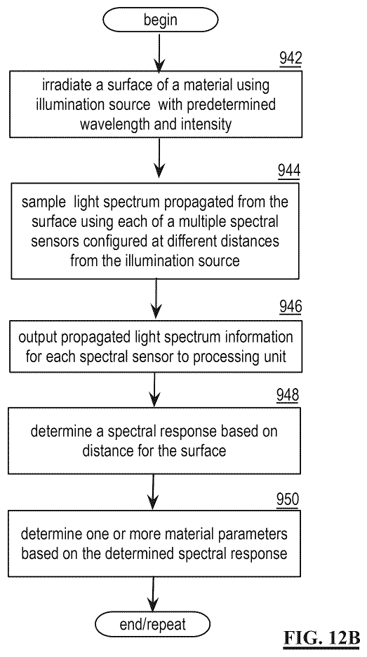

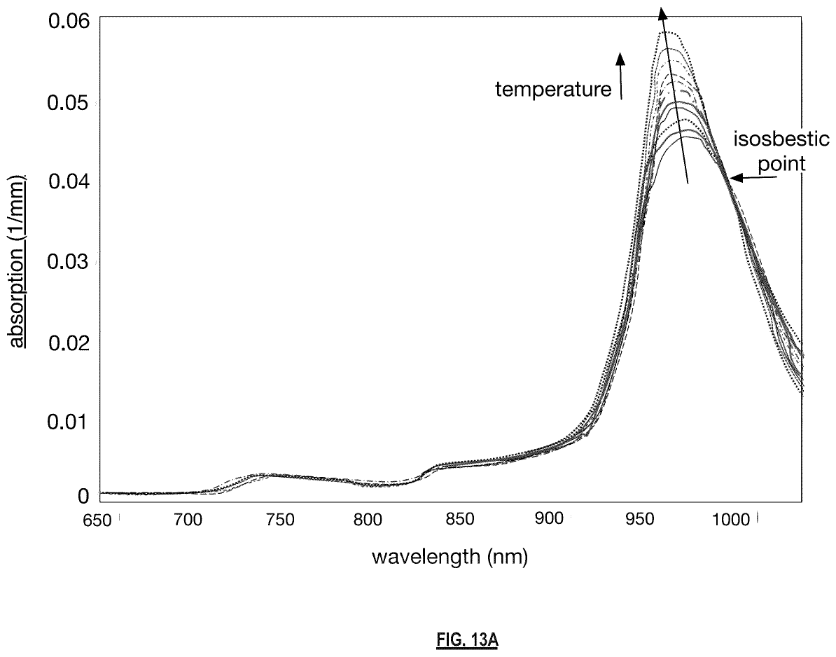

FIG. 12A provides an illustration of a spectral sensing system incorporating multiple spectral sensors 682 with associated illumination sources 680. In an example, multiple spectral sensors 682 can be configured in an array, with illumination sources 680 configured to provide illumination relatively evenly around the array. In a specific example of implementation, the illumination sources 680 are configured in a ring around the array, the sensors in the center of the array having a different spectral response than the sensors in the edges because of their relative distance to the illumination sources 680. In another example, the illumination sources 680 are evenly distanced from the edges of the array, in the form of a rectangle or square. In another specific example, the spectral sensors 682 are configured to alternate height of the sensor relative to the mount, such that a lowest and highest mounted spectral sensor are adjacent to each other on alternating spectral sensors. In yet another example, a single sensor wedge (such as sensor wedge 666 of FIG. 11B) is used, with illumination distributed around the single sensor wedge 666. In yet another alternative example, either of the illumination sources 680 or any of spectral sensors 682 can be mechanically moved to adjust its relative distance from illumination sources 680 or a sample being measured, with the movement performed through a series of steps and a measurement performed at each step.

In a specific example of implementation, one or more collimating elements are configured proximate to the sensor wedge(s) 666 to isolate spatial information from a sample being observed/measured. In an example, the one or more collimating elements can be configured to reduce incident light from leading to an adjacent spectral sensor 682 of the sensor wedge 666.

FIG. 12B is a flowchart of a method for determining the spectrophotometric parameters of a material. The method begins at step 942, with a material, such as an area of skin, being irradiated by one or more illumination sources, where each of the one or more illumination sources is configured to provide light within a predetermined range of optical wavelengths and are configured to irradiate light directly onto the area of skin. In an example, the illumination sources are additionally configured to provide light across the predetermined range of optical wavelengths at a predetermined intensity. The method continues at step 944 with each of a plurality of spectrometers sampling a received light spectrum from the material, where each of the plurality of spectrometers includes a plurality of interference filters overlaying one or more optical sensors and each of the optical sensors has a sensing range within a predetermined range of optical wavelengths. In an example, each of the plurality of spectrometers is configured to capture light emitted from the material and each of the one or more spectrometers is positioned a different distance from the material and each spectrometer.

At step 946 each of the plurality of spectrometers output information representing the response of the spectrometer to one or more modules of a processing device and at step 948 the processing device determines a spectral response for each of the plurality of spectrometers. In an optional step 950, the one or more material parameters can be determined at least partially based on a comparison of the response from the one or more spectrometers with a reference response, where the reference response is one or more of a response database, a comparison with an earlier stored response and a classification engine (such as a neural network or cognitive computing engine). In an example, the material is a translucent or partially translucent material, such as skin or tissue. In another example, the material is a liquid, such as an aqueous or nonaqueous solution, a colloid having dispersed molecules or polymolecular particles and/or a semi-solid, such as a gel. In yet another example, the material is at least partially gaseous, such as gas contained in a translucent container.

Referring again to FIG. 11, since light of different wavelengths can penetrate deeper into skin or tissue, by measuring a spectrum at different distances from the skin or tissue surface a comparison of the spectral response can be used to determine parameters of the skin or tissue at those different skin or tissue depths. Referring to FIG. 12D, in an example when skin is the material being observed/measured, the skin hydration at different depths of the skin can be evaluated based on the spectral response at those different depths. For example, if differential detection using three wavelengths 1720, 1750, and 1770 nm is being used, the lipid vibrational bands between these water absorption bands can be used to approximate hydration levels and skin sebum in skin at each of the different depths. Accordingly, the accuracy and/or precision of the measurement can be enhanced, while providing a better understanding of the hydration and presumably its effect on a user's health.

In another example, physiological parameters associated with other health conditions can be evaluated in blood and tissue. Examples include but are not limited to lactate, carbon dioxide (CO₂) and/or carbon monoxide (CO) level, hemoglobin content, along with glucose and/or insulin levels.

Physiological parameters associated with various health conditions, such as diabetes, cancer and asthma, along with the physiological parameters associated with health affecting habits such as smoking and drug use can all be evaluated. In an example, a health care professional can use the determined physiological parameters to evaluate, track and treat health conditions to aid in the treatment of disease and/or overall health. Moreover, in an example the determined physiological parameters can used in the diagnosis of disease, the adjustment of dosage of pharmaceuticals and defining of insurance coverage. In an example, determined physiological parameters can be compared to reference parameters, such as one or more of a database of physiological parameters, a comparison with earlier stored physiological parameters and/or comparison to 3^{rd} party physiological parameters using a classification engine (such as a neural network or cognitive computing engine).

In another specific example of implementation and operation, physiological parameters can be subject to relatively continual measurement. In an example, the physiological parameters can be evaluated during travel in an automobile, motorcycle, airplane, etc. for safety and health reasons. For example, physiological parameters such as alcohol concentration in blood, SpO2, SpCO, heart rate, and PPG could be continually monitored, with a signal or other notification being transmitted when predetermined threshold are exceeded. In an example related to CO poisoning, an automated notification can be particularly useful to warn people who are sleeping or would otherwise and may not be aware of an increase in CO, for example in an underground mine or another environment where the risk of CO poisoning is high.

The notification could indicate one or more health risks, such as excessive alcohol levels in the blood, a dangerous heart arrhythmia, Carbon Monoxide (CO) poisoning or indication associated with heart attack.

In an example, the notification can include one or more of a visual display on a screen, an audible sound or a vibration, any of which can be integrated in one or more of a driving wheel, a seat and a helmet. Example notification mechanisms include haptic sensors and/or haptic feedback devices, such as eccentric rotating mass (ERM) actuators and linear resonant actuators (LRAs). In another example, the notification can initiate the safe automated stoppage of a vehicle.

In an example, physiological parameters associated with health conditions can be detected. In a specific example of operation and implementation, by monitoring SpO2, heart rate and/or photoplethysmogram (PPG), a spectrophotometric system can provide an alert when a health condition is indicated. For example, when one of SpO2, heart rate, PPG levels or a combination of the same are at a level indicating a possibility of sleep apnea, an alert can automatically provide for use in a visual display, an audible sound or vibrations. In an example, all or a part of a spectrophotometric system can be integrated in a wearable device or smart clothing, such as sleepwear and sleeping gowns. In an example of implementation, the system can be configured to transmit a notification to a user prompting the user to wake up or to a health care assistant who can then provide treatment.

In a related example, when one of SpO2, heart rate, PPG levels or a combination of the same are at a level indicating a risk of edema, an alert can automatically provide to a user to take appropriate action. Again, all or a part of a spectrophotometric system can be integrated in a wearable device or smart clothing, such as compression stockings or leggings.

In another example, physiological parameters associated with physical activities, such as sports, can be detected. In an example, by monitoring SpO2, heart rate, PPG a spectrophotometric system can provide a continuous indication of the levels of each. In an example, all or a part of a spectrophotometric system can be integrated in wearable devices, smart clothing or training equipment such as watches or patches. In a specific example of implementation, the system can be configured for use during underwater diving, where it can measure the SpO2 levels of a diver and provide an alert if the SpO2 values drops below a predetermined threshold.

In an example, an alert can be sent to one or more of a user, a diving instructor or the captain of a dive boat. In another specific example, a spectrophotometric system can provide a continuous indication of physiological parameters to athletes training at high altitudes such as climbers, hikers and mountain bikers. In an example, the physiological parameters can provide information relating to a user's reaction to altitude and can assist an evaluate of training regimes by, for example, monitoring improvements in oxygenation due to red blood cell levels. In a related example a spectrophotometric system can be combined with GPS or other means of geolocation to monitor the position of the user when physiological parameters are being monitored. In an example, location information can be used to log how deep a diver was or how high a climber was when certain physiological parameters were measured in order to optimize a training regime or to prevent associated health risks.

Since the spectrometer systems of FIGS. 11A, 11B and 12A are relatively inexpensive while being potentially highly mobile, these systems can provide substantial economic benefits in health care delivery and the systems lend themselves easily to remote health care administration. In a related example, such a spectrometer system can be inherently computer and cloud-based, such that feedback (such as, for example drug dosage) could be nearly immediate and can also be tracked automatically. In another example, data collected can be easily shared with researchers and other interested parties in order rapidly train expert systems and artificial intelligence engines for the advancement of treatments and epidemiological analysis.

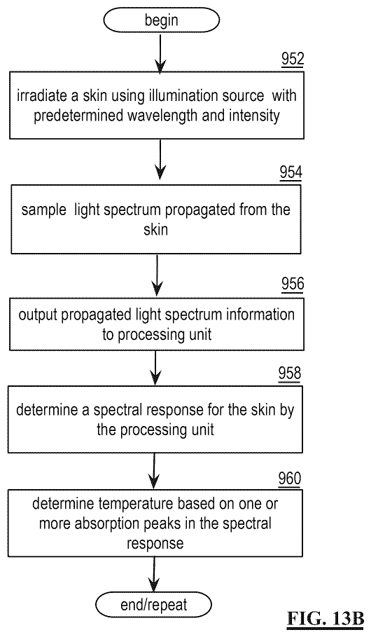

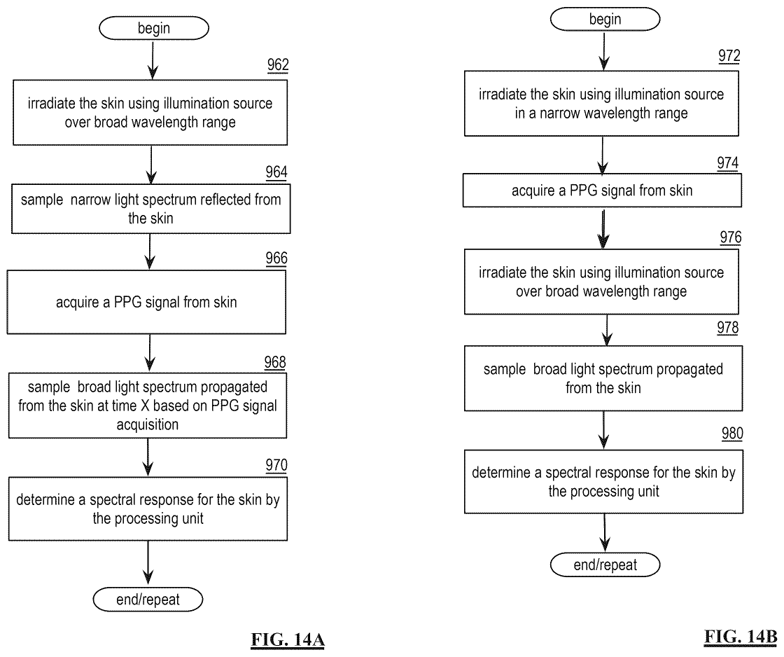

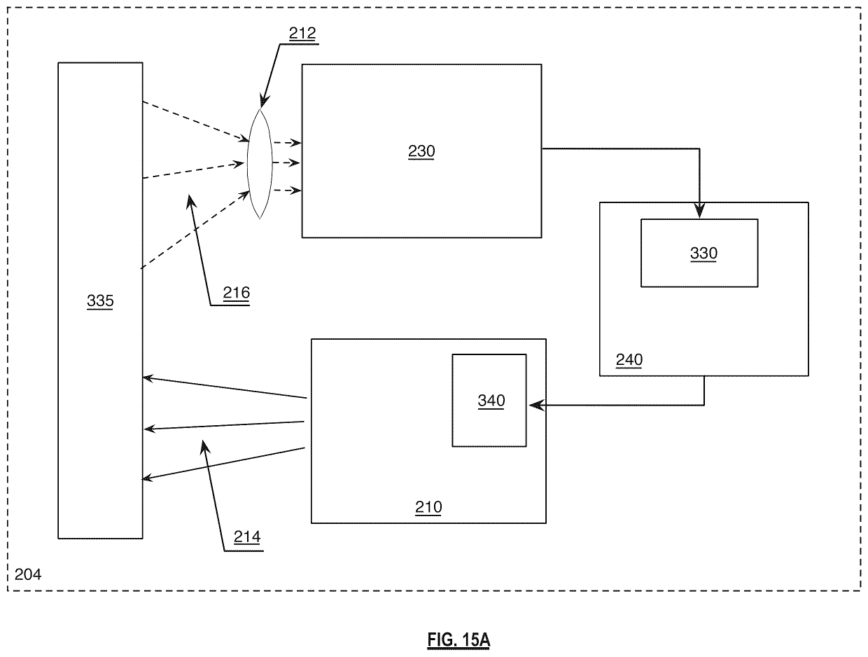

FIG. 13A illustrates Isosbestic Points for a water absorption peak as a function of temperature. In the illustration, the water absorption peak (around 970 nm) is shown to shift at the Isosbestic Point according to the temperature. In an example, broadband diffuse optical spectroscopy, based on opposing shifts in near-infrared (NIR) water absorption spectra, reflect the temperature and macromolecular binding states of skin/tissue. In a further example, thermal and hemodynamic (i.e. oxy- and deoxy-hemoglobin concentration) changes can be measured simultaneously and continuously in skin, such that the opposing shifts can be used for non-invasive, co-registered measurements of absolute temperature and hemoglobin parameters in skin and thick tissue. In an example, the water absorption peak and potentially other absorption peaks for other tissue constituents can be used to improve thermal diagnostics and therapeutics.

FIG. 13B is a flowchart of a method for determining the temperature of skin or other tissue using a spectrophotometer. The method begins at step 952, with an area of skin being irradiated by one or more illumination sources, where each of the one or more illumination sources is configured to provide light within a predetermined range of optical wavelengths and are configured to irradiate light directly onto the area of skin. In an example, the illumination sources are additionally configured to provide light across the predetermined range of optical wavelengths at a predetermined intensity. The method continues at step 954 with one or more spectral sensors sampling a received light spectrum from the area of skin, where each of the one or more spectrometers includes a plurality of interference filters overlaying one or more optical sensors and each of the one or more spectrometers has a sensing range within a predetermined range of optical wavelengths. In an example, each of the one or more spectrometers is configured to capture light emitted from the area of skin and each of the one or more spectrometers is positioned a predetermined distance from at least one illumination source.

At step 956 the one or more spectrometers output information representing the response of the one or more spectrometers to one or more modules of a processing device and at step 958 the processing device determines a spectral response for at least a portion of the area of skin. The method continues at step 960, with the processing device using the measured spectrum, to determine the temperature of the area of skin. In an example, the temperature is determined based on the absorption peak of a known reference. In another example the temperature is determined based on reference to another temperature gathering device. In yet another example the temperature is determined based on a combination of a reference absorption peak and another temperature gathering device. In still another example the method of FIG. 13B is used for relatively continuous monitoring of an absorption peak in order to provide changes in temperature over a period of time.

In a related example of implementation and operation, both a spectroscopic model such as a chemometric model and the Isosbestic point can be used to analyze various parameters of a sample. In another example, a preprocessed spectrum is used, such as a derivative of the spectrum. I another example, the spectrum is first resolved from a spectral PPG signal, consisting of a spectrum collected from the amplitudes of a PPG signal out of each spectral filter; since the PPG signals in first order correlate only to contributions from blood, the spectrum refers to the water in blood. The spectrum of a PPG signal may be less affected by other confounding factors, to determine the temperature dependance of the water absorption peak.

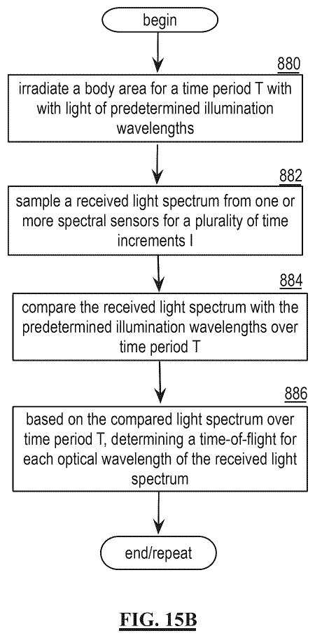

FIG. 14A is a flowchart of a method for collecting a photoplethysmogram using a spectrophotometer. A photoplethysmogram (PPG) is an optically obtained plethysmogram that can be used to detect blood volume changes in the microvascular bed of tissue. In an example, a PPG can be obtained by using a pulse oximeter to measures changes in light absorption to measure heart rate estimation and pulse oxymetry readings. In another example, a PPG signal includes a second derivative wave, analysis of which can be used to evaluate various cardiovascular-related diseases such as atherosclerosis and arterial stiffness. In another example, the second derivative wave of PPG signal can also assist in early detection and diagnosis of various cardiovascular illnesses that may possibly appear later in life.

In a specific example of implementation and operation, photoplethysmogram (PPG) signals can be used to replace electrocardiogram (ECG) recordings for the extraction of heart rate variability (HRV) signals. In an example, a PPG signal comprises pulsatile (AC) and superimposed (DC) components, where the AC component is provided by cardiac synchronous variations in blood volume that arise from heartbeats. The DC component is shaped by respiration, sympathetic nervous system activity, and thermoregulation and in an example, the AC component depicts changes in blood volume, which are caused by cardiac activity and depend on the systolic and diastolic phases.

The method begins at step 962, with an area of skin being irradiated by one or more illumination sources, where each of the one or more illumination sources is configured to provide light within a predetermined range of optical wavelengths and are configured to irradiate light directly onto the area of skin. In an example, the illumination sources are additionally configured to provide light across the predetermined range of optical wavelengths at a predetermined intensity. The method continues at step 964 with one or more spectral sensors sampling a received light from the area of skin in a narrow wavelength range, where each of the one or more spectrometers includes a plurality of interference filters overlaying one or more optical sensors and each of the one or more spectrometers has a sensing range within a predetermined range of optical wavelengths. In an example, each of the one or more spectrometers is configured to capture light emitted from the area of skin and each of the one or more spectrometers is positioned a predetermined distance from at least one illumination source.

At step 966 a photo-plethysmogram (PPG) is obtained while the one or more spectral sensors are sampling the received light from the area of skin in the narrow wavelength range. The PPG can be obtained by measuring the changes in light absorption at the narrow sampling wavelength range during one or more cardiac cycles. The method continues at step 968, with the one or more spectral sensors sampling received light from the area of skin in a broader wavelength range at a time X dictated by the PPG sampling. In an example, the broader wavelength can include all the available wavelength channels of the one or more spectral sensors or a portion thereof. The method continues at step 970, with the processing device determining a spectral response for the skin.

FIG. 14B is a flowchart of a method for collecting a photoplethysmogram (PPG) using a spectrophotometer. The method begins at step 972, with an area of skin being irradiated by one or more illumination sources in a narrow wavelength range, where each of the one or more illumination sources is configured to provide light within a predetermined range of optical wavelengths and are configured to irradiate light directly onto the area of skin. In an example, the illumination sources are additionally configured to provide light across the predetermined range of optical wavelengths at a predetermined intensity. At step 974 a PPG signal is obtained while one or more spectral sensors are sampling the received light from the area of skin in the narrow wavelength range. Each of the one or more spectrometers includes a plurality of interference filters overlaying one or more optical sensors and each of the one or more spectrometers has a sensing range within a predetermined range of optical wavelengths. In an example, each of the one or more spectrometers is configured to capture light emitted from the area of skin and each of the one or more spectrometers is positioned a predetermined distance from at least one illumination source. The PPG can be obtained by measuring the changes in light absorption during one or more cardiac cycles. The method continues at step 976, with the area of skin being irradiated by the one or more illumination sources in a broad wavelength range. The method continues at step 978 with the one or more spectral sensors sampling the received light from the area of skin, and then continues at step 980, with the processing device determining a spectral response for the skin.

FIG. 15A is a block diagram of a system for a measuring range incorporating a spectroscopy device 204. In an example, illumination source(s) 210 provide modulated illumination (214) of skin sample 335 controlled by control circuit 340. In an example, light 212 propagated from the skin sample 335 is collected via lens 212 at spectral sensor array 230 and a spectral response is output to computing module 330 of computing device 240. In an example, measuring the phase angle of the wavelengths of light 216 received at spectral sensor array 230 enables the calculation of the distance the light traveled at each of the measured wavelengths using a time-of-flight approach. In another example, the change in frequency of wavelengths of light at the spectral sensor array 230 relative to the frequency at the illumination source(s) 210 are used to calculate a Doppler shift for each of the measured wavelengths.

In an example, a device is configured to measure phase shifts in light reflecting from skin (assuming the phase properties of the illumination source are known) and determine the depth of travel by the light inside the skin using a time-of-flight approach. In an example, the information on skin depth can be used to create tomography-like information to measure health parameters. In another example, a device is configured to measure a Doppler shift for light being collected at various wavelengths at a spectrometer, by monitoring a change in frequency of light at the spectrometer relative to the frequency at the illumination source. In an example, Doppler shift can be used to determine photoplethysmogram (PPG) signal, heart rate and blood flow speed.

In a specific example of implementation and operation, a device includes one or more illumination sources, where each of the one or more illumination sources is configured to provide light within a predetermined range of wavelengths, and the illumination sources is configured to irradiate light directly onto skin or tissue. In an example, at least one of the one or more illumination sources is adapted to be modulated. In an example, the device includes one or more spectrometers, wherein each of the one or more spectrometers includes a plurality of interference filters overlaying one or more optical sensors and each of the one or more spectrometers has a sensing range within a predetermined range of optical wavelengths. In an example, each the spectrometers are configured to capture light emitted from the skin and are positioned a predetermined distance from at least one illumination source of the one or more illumination sources.

In a specific example, the device includes a first module of a processor configured to receive an output from the one or more spectrometers and a second module of the processor is configured to determine a time-of-flight based on modulation of at least one of the one or more illumination sources adapted to be modulated and the output from the one or more spectrometers. In an example, the one or more illumination sources is adapted to be modulated in a single wavelength. In another example, blood flow and/or photoplethysmogram (PPG) signals are determined based at least partially on the determined time-of-flight.

In a specific example of implementation and operation, a device includes one or more spectrometers, where each of the one or more spectrometers includes a plurality of interference filters overlaying one or more optical sensors and each of the one or more spectrometers has a sensing range within a predetermined range of optical wavelengths. In an example, each of the spectrometers is configured to capture light emitted from skin or tissue and is positioned a predetermined distance from at least one illumination source of one or more illumination sources. In an example, each of the one or more illumination sources is configured to provide light within a predetermined range of wavelengths, and the illumination sources is configured to irradiate light directly onto the skin or tissue. In an example, at least one of the one or more illumination sources is adapted to be modulated and the predetermined range of wavelengths for the at least one of the one or more illumination sources is adapted to be modulated in substantially the same wavelengths as a sensing range for the plurality of spectrometers.

In another example, at least one of the one or more illumination sources is adapted to be modulated subject to a controller to produce a controlled modulation. In an example, the controlled modulation is used to additional information at the spectrometer(s).

FIG. 15B is a flowchart of a method for determining time-of-flight using a spectrophotometer. The method beings at step 880, by irradiating, using one or more illumination sources, a body area with light for a period of time T, where each of the one or more illumination sources is configured to provide light within a predetermined range of optical wavelengths. The method continues at step 882, by sampling a received light spectrum from one or more spectral sensors for a plurality of time increments I, wherein the sum of the plurality of time increments I equal to the period of time T, where each of the one or more spectrometers includes a plurality of interference filters overlaying one or more optical sensors and each of the one or more spectrometers has a sensing range within a predetermined range of optical wavelengths and is configured to capture light emitted from the body area. In an example each of the one or more spectrometers is positioned a predetermined distance from at least one illumination source of the one or more illumination sources.

The method then continues at step 884, when a processor compares the received light spectrum with the predetermined illumination wavelengths over at least a portion of time period T and continues at step 886, when based on the compared light spectrum over time period T, a time-of-flight is determined for each optical wavelength of the predetermined range of optical wavelengths of at least one illumination source of the one or more illumination sources. In an example, the time-of-flight information for the optical wavelengths can be used to determine characteristics of the body area, including the tissue at relative depths in the body area.

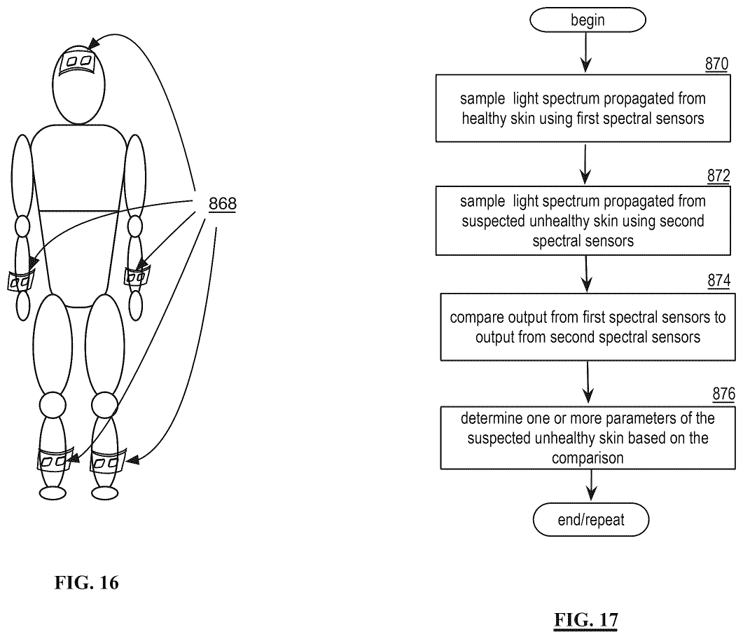

FIG. 16 illustrates a system for monitoring blood pressure using multiple spectral sensors 868. In an example, spectral sensor modules 868 are placed in different positions of the body of a user, with each device acquiring a PPG signal using the spectral sensors embodied in spectral sensor modules 868. In an example, by checking the differential in timing of the PPG signals from each of the spectral sensors, the acquired PPG signals can be used to measure and monitor blood pressure. In a specific example of implementation and operation, a system for measuring optical response from skin includes a plurality of spectrometers, where each of the plurality spectrometers includes a plurality of interference filters overlaying one or more optical sensors and each of the plurality of spectrometers has a sensing range within a predetermined range of optical wavelengths configured to capture light emitted from the skin. Each of the spectrometers further includes one or more illumination sources, where each of the illumination sources is configured to provide light within a predetermined range of optical wavelengths and is configured to irradiate light directly onto skin. In an example, each spectrometer is positioned a predetermined distance from at least one illumination source. In another example, the relative shape of the spectral photoplethysmogram (PPG) signals can be used to correlate to blood pressure. In a specific example, the differential of the PPG signals is used.

In an example, one or more modules of a computing device associated with each of the spectrometers is configured to transmit an output from the associated spectrometer of the plurality of spectrometers to one or more modules of a system computing device configured to receive the output from each spectrometer of the plurality of spectrometers. In a specific example of implementation and operation, the one or more modules of the system computing device are configured to compare the output from each spectrometer of the plurality of spectrometers to other spectrometers of the plurality of spectrometers to produce a comparison. In a related example, the one or more modules of the system computing device are also configured to monitor the output from each computing device associated with a spectrometer and produce a measurement of one or more physiological attributes. In an example, the physiological attributes can include blood pressure, where the blood pressure is determined based on a comparison of PPG signals from each of spectrometers. In an example, the output from each spectrometer is representative of a PPG signal. In another example the system computing device is the computing device associated with a spectrometer and in a related example, the plurality of spectrometers are wirelessly connected using a mesh network.

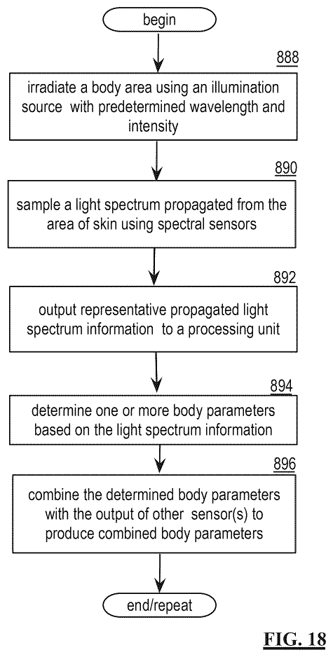

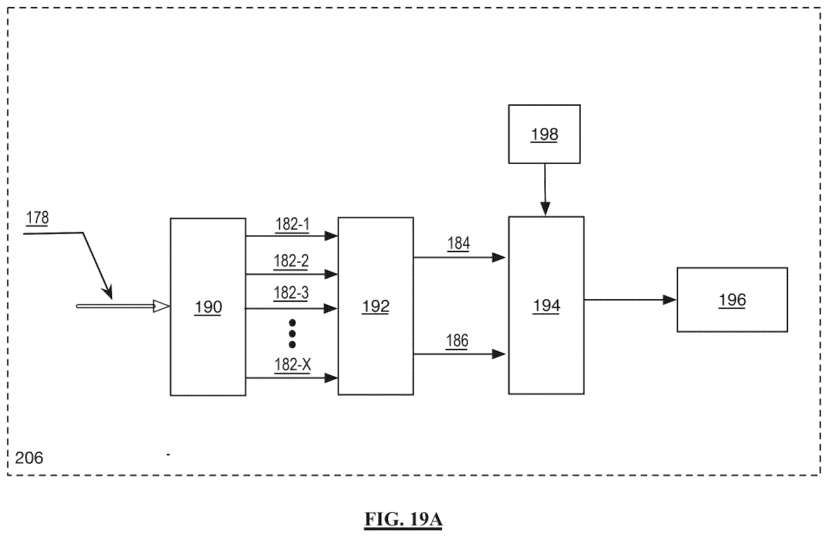

FIG. 17 is a flowchart illustrating an example method for monitoring wound healing using a spectral sensor. The method begins at step 870, with a first one or more spectral sensors sampling a received light spectrum from a known healthy area of skin, where each of the first one or more spectrometers includes a plurality of interference filters overlaying one or more optical sensors and each of the one or more spectrometers has a sensing range within a predetermined range of optical wavelengths. In an example, each of the first one or more spectrometers is configured to capture light emitted from the healthy area of skin. The method continues at step 872, with a second one or more spectral sensors sampling a received light spectrum from a known or suspected unhealthy area of skin, where each of the second one or more spectrometers includes a plurality of interference filters overlaying one or more optical sensors and each of the second one or more spectrometers has a sensing range within a predetermined range of optical wavelengths. In an example, each of the second one or more spectrometers is configured to capture light emitted from the suspected unhealthy area of skin. In a specific example, the suspected unhealthy area of skin can include a wound that is being monitored for healing. In another example, suspected unhealthy area of skin can include a diseased area of skin being monitored for treatment and/or status. In yet another example, the suspected unhealthy area of skin can include a symptom of a larger disease, such diabetes or phlebitis and the monitoring of the area of skin informs progression of the larger disease.

The method continues at step 874, where one or more modules of a processing device compare an output from each of the first and second spectral sensors to produce a comparison. The method then continues at step 876, with one or more modules of a processing device determining one or more parameters of the suspected unhealthy skin based on the comparison. In an example, determining the parameters can include a further comparison to a reference, such as an earlier measurement of the suspected unhealthy skin. In another example, the differential between the known healthy skin and the suspected unhealthy skin can be used for evaluation and classification using a reference database. In yet another example, the differential between the known healthy skin and the suspected unhealthy skin can be analyzed using a trained neural network or cognitive computing engine to provide an assessment and/or suggest treatment options. In a specific example of implementation and operation, the monitoring can be used to inform treatment of the suspected unhealthy skin, such as determining a change in treatment or confirming the continuation of a treatment regimen.

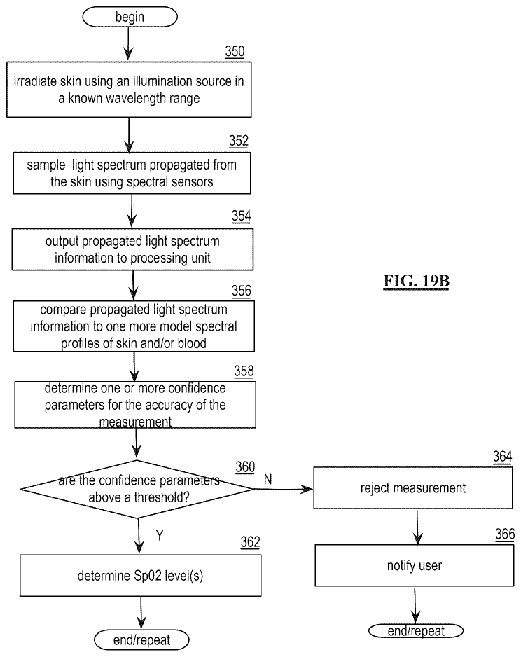

FIG. 18 is a flowchart illustrating an example method for using a spectral sensor to augment other sensors. The method begins at step 888, with a body area being irradiated by one or more illumination sources. In an example, each of the one or more illumination sources is configured to provide light within a predetermined range of optical wavelengths and are configured to irradiate light directly onto the body area. In an example, the illumination sources are additionally configured to provide light across the predetermined range of optical wavelengths at a predetermined intensity. In an alternative example, the illumination source is natural light, such as direct or indirect sunlight. The method continues at step 890 with one or more spectral sensors sampling a received light spectrum from the body area, where each of the one or more spectrometers includes a plurality of interference filters overlaying one or more optical sensors and each of the one or more spectrometers has a sensing range within a predetermined range of optical wavelengths. In an example, each of the one or more spectrometers is configured to capture light emitted from the area of skin and each of the one or more spectrometers is positioned a predetermined distance from at least one illumination source.

The method continues at step 892 when the one or more spectrometers output information representing the optical response of the one or more spectrometers to one or more modules of a processing device and at step 894 the processing device determines one or more body parameters for at least a portion of the body area. The method continues at step 896, with the body parameters determined based on the optical response being combined with the output of one or more other sensors to produce a combined result. In an example, the body parameters are one or more biometric indicators, with the output of the spectrophotometric and other sensors being combined to provide enhanced biometric identification.

In another example, output of the spectrophotometric sensors is combined with skin resistivity sensor measurements to provide additional parameters, such as heart rate, while the skin resistivity sensor is used to measure sweat production. In a related example, the output of the spectrophotometric sensor is used alongside the output of a second sensor capable of measuring the heart rate. In an example, the heart rate measurement from the second sensor is used to improve the reliability of the spectrophotometric sensors for determining biometric parameters. In an example, the second sensor output is used to clean the output of the spectrophotometric sensors by removing artifacts produced by heart rate. In another example, the second sensor output is used to cross-check a heart rate signal determined based on the output of the spectrophotometric sensors. Examples of second sensors capable of measuring heart rate include ECG sensors and spectral devices working in the near-infrared (NIR) wavelengths. Examples of combined parameters with potential for improvement with are SpO₂, SpCO₂, SpCO and PPG.

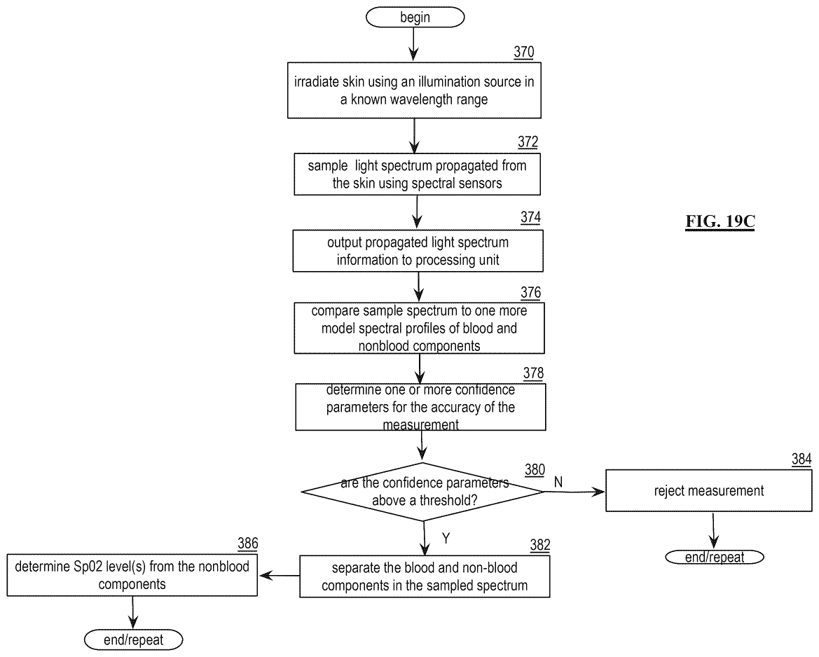

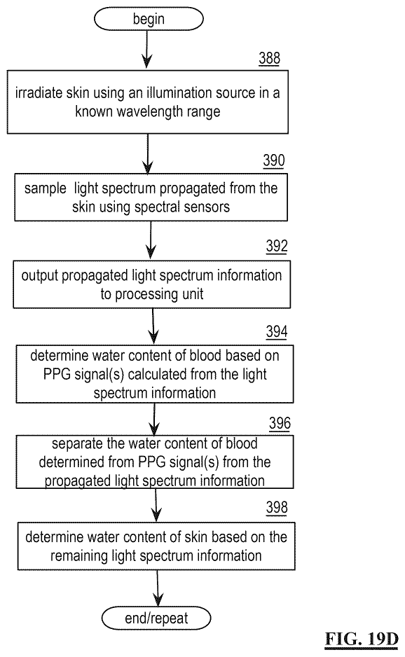

FIG. 19A provides an illustration of a spectral sensor system 206 that uses photoplethysmogram (PPG) signals to determine sample parameters. In an example, the collection of sample parameters using a spectroscopic model from skin or tissue can result in erroneous measurements. Potential error sources include unintended motion of the sensors or sample, along with compounding factors such as body hair, nail polish, tattoos, carboxyhemoglobin, etc. In an example, SpO₂ is normally calculated using a two-wavelength approach, where the SpO₂ signal is calculated or correlated using a weighted response of a perfusion index red (PIred) and perfusion index infrared (PIir) (PI is a perfusion index, taken from the AC/DC signal of a PPG signal). When using PI number there is no way to determine whether a given measurement is faulty or otherwise compromised. Using the spectral sensor, a confidence image can be generate and used to confirm the accuracy of a measurement. In an example, the confidence image can be compared to a known spectral profile of skin or blood to confirm a valid measurement.

In an example, one or more spectral sensors 190 are used to determine one or more PPG signals PPG_{1,} PPG_{2,} PPG_{3,} through PPG_{N} (182-1 to 182-x) from a sample. In an example, spectral sensor 190 is configured to receive light 178 propagated from the sample and output PPG signals to a processor, such as a digital signal processor, which is configured to output an AC component 184 and DC component 186 for each of one or more of 182-1 to 182-x to a processing device. In an example, the processing device is configured to use the AC/DC components 184 and 186 of the one or more PPG signals 182-1 to 182-x to determine a desired parameter for the sample.

FIG. 19B is a flowchart illustrating an example method for using a spectrophotometer to conform the validity of sample analysis. The method begins at step 350, with a sample of skin or tissue being irradiated with one or more illumination sources of a known wavelength range. The method continues at step 352, with a light spectrum propagated from the sample being sampled using one or more spectral sensors and continues at step 354 with the propagated light spectrum information being output to a processing unit. The method continues at step 356, where the processing unit is used to compare the propagated light spectrum information to one or more model profile spectra of skin and/or blood. At step 358 the processing unit determines confidence parameters based on the comparison of the propagated light spectrum information to the one or more model profile spectra of skin and/or blood and continues at step 360, with the processing unit determining whether the confidence parameters meet or exceed a confidence threshold. When the confidence threshold is not met, the method continues at step 364 with the processing unit being used to reject the measurement. In an optional step 366, the processing unit can initiate a notification to a user that the measurement has been rejected, so that a user can take appropriate action, such as manipulating the measurement device (vis. tightening or resecuring a restraint). When the confidence threshold is met, the method continues at step 362, with one or more parameters, such as SpO₂ being calculated.

FIG. 19C is a flowchart illustrating another example method for using a spectrophotometer to confirm the validity of sample analysis. The method begins at step 370, with a sample of skin or tissue being irradiated with one or more illumination sources of a known wavelength range. The method continues at step 372, with a light spectrum propagated from the sample being sampled using one or more spectral sensors and continues at step 374 with the propagated light spectrum information being output to a processing unit. The method continues at step 356, where the processing unit is used to compare the propagated light spectrum information to one or more model profile spectra of blood and nonblood components, such as the blood and nonblood components of skin or tissue. At step 378 the processing unit determines confidence parameters based on the comparison of the propagated light spectrum information to the one or more model profile spectra of blood and nonblood components and continues at step 380, with the processing unit determining whether the confidence parameters meet or exceed a confidence threshold. In an example, the confidence parameters can be calculated using the residuals from partial least squares path modeling (PLS-PM) or partial least squares structural equation modeling (PLS-SEM). In another example, the confidence parameters can be calculated using Hotelling's T-squared distribution (T2). In an example, when the spectrum shows large residuals, the measurement may be inconsistent with the model.

When the confidence threshold is not met, the method continues at step 384 with the processing unit being used to reject the measurement. In an optional step the processing unit can initiate a notification to a user that the measurement has been rejected and/or prompting user action. In another example, background light may be detected, with a user being notified instructed to tightening or resecure a restraint, such as a watch band. In another example, low blood content or low-perfusion may be measured in the spectrum, whereby a user can be instructed to perform a brief period of physical activity to prompt more blood circulation, or being instructed to re-perform the measurement in a warmer location. When the confidence threshold is met, the method continues at step 382, where the processing unit is used to separate the blood and nonblood components of the sample spectrum. At step 386 the processing unit is used to calculate SpO₂ based on the blood components determined at step 382. In an example, continuous data capturing fewer data points could be available for averaging or tracking, but the data points measured will be more accurate, leading to, for example, more accurate SpO₂ readings. The method of 19B and 19C can also be used for other parameters. In a specific example, SpO₂ is continuously measured and poor data is rejected, so that over time, sufficient good data are available for continuous monitoring of SpO₂.

Dermal H₂O (water) can reside multiple millimeters deep in skin tissue. Referring to FIG. 13A, dermal water can be measured using a wearable mobile device on skin, such as a patch or a wristwatch using, for example, an NIR spectral sensor. In an example, body water variations may be determined by analyzing longitudinal measurements of dermal water and using a model (such as an artificial intelligence (AI) model) to predict body water variations. In an example, a body water model can also consider parameters such as age, gender, motion, temperature and heart rate.

FIG. 19D is a flowchart illustrating an example method for using a spectrophotometer to measure the water content of skin or tissue. In some body locations water present in blood can interfere with water in skin measurements. For example, when numerous blood vessels are present just beneath the surface of skin, blood in the vessels can interfere with dermal water measurements.

In an example, spectroscopic measurement associated with PPG signals may be used to first determine the contribution of water content of blood and then differentiate the water content of blood from water measured in the tissue in and around the blood vessels. The method of FIG. 19D begins at step 388, with a sample of skin or tissue being irradiated with one or more illumination sources of a known wavelength range. In an example, the one or more illumination sources can be configured in accordance with the methods illustrated in FIG. 14A and/or FIG. 14B. In another example, the illumination sources can be configured to provide narrowband illumination for PPG signal acquisition/calculation in as well as wideband illumination over a time period. In yet another example, the illumination sources can provide wideband illumination. The method continues at step 390, with a light spectrum propagated from the sample being sampled using one or more spectral sensors. In an example, the sampling can include both one or more narrowband samples and a wide band sample over a period of time. The method continues at step 392 with the propagated light spectrum information being output to a processing unit.

The method continues at step 394, where the processing unit is used to determine water content in blood using one or more PPG signals calculated using the propagated light spectrum information. In an example, the PPG signals can be obtained by measuring the changes in light absorption during one or more cardiac cycles. The method continues at step 396, with the water content in the blood determined based on one or more PPG signals is separated from the sampled light spectrum information. In an example, the separating can be based on subtracting the spectrum contribution of the determined PPG signal(s) from the propagated light spectrum information. In another example the separating can involve the use of more sophisticated mechanisms, such as an expert system and/or artificial intelligence engine. The method then continues at step 398, where the remaining light spectrum information is used to determine the water content of the skin.

In an example, a plurality of spectral sensors at different distances from an illumination source may be used to determine water levels at different depths of skin tissue. In an example, the plurality of spectral sensors can be used to provide more accurate water measurements. In another example, the plurality of spectral sensors can be used to correct for water content from blood as compared to water content from dermal water. In yet another example, water content in blood calculated using PPG spectroscopy can be used to diagnose other medical issues. In an example, a plurality of water measuring sensors on the body can be used according to one or more models to predict body water levels or body water level variations. In another example, a selfie spectral camera, or face-targeting spectral camera can be used to determine facial hydration levels useful to advise the use of specific hydrating cremes and/or other treatments.

It is noted that terminologies as may be used herein such as bit stream, stream, signal sequence, etc. (or their equivalents) have been used interchangeably to describe digital information whose content corresponds to any of a number of desired types (e.g., data, video, speech, text, graphics, audio, etc. any of which may generally be referred to as ‘data’).

As may be used herein, the terms “substantially” and “approximately” provide industry-accepted tolerance for its corresponding term and/or relativity between items. For some industries, an industry-accepted tolerance is less than one percent and, for other industries, the industry-accepted tolerance is 10 percent or more. Other examples of industry-accepted tolerance range from less than one percent to fifty percent. Industry-accepted tolerances correspond to, but are not limited to, component values, integrated circuit process variations, temperature variations, rise and fall times, thermal noise, dimensions, signaling errors, dropped packets, temperatures, pressures, material compositions, and/or performance metrics. Within an industry, tolerance variances of accepted tolerances may be more or less than a percentage level (e.g., dimension tolerance of less than +/−1%). Some relativity between items may range from a difference of less than a percentage level to a few percent. Other relativity between items may range from a difference of a few percent to magnitude of differences.

As may also be used herein, the term(s) “configured to”, “operably coupled to”, “coupled to”, and/or “coupling” includes direct coupling between items and/or indirect coupling between items via an intervening item (e.g., an item includes, but is not limited to, a component, an element, a circuit, and/or a module) where, for an example of indirect coupling, the intervening item does not modify the information of a signal but may adjust its current level, voltage level, and/or power level. As may further be used herein, inferred coupling (i.e., where one element is coupled to another element by inference) includes direct and indirect coupling between two items in the same manner as “coupled to”.

As may even further be used herein, the term “configured to”, “operable to”, “coupled to”, or “operably coupled to” indicates that an item includes one or more of power connections, input(s), output(s), etc., to perform, when activated, one or more its corresponding functions and may further include inferred coupling to one or more other items. As may still further be used herein, the term “associated with”, includes direct and/or indirect coupling of separate items and/or one item being embedded within another item.

As may be used herein, the term “compares favorably”, indicates that a comparison between two or more items, signals, etc., provides a desired relationship. For example, when the desired relationship is that signal 1 has a greater magnitude than signal 2, a favorable comparison may be achieved when the magnitude of signal 1 is greater than that of signal 2 or when the magnitude of signal 2 is less than that of signal 1. As may be used herein, the term “compares unfavorably”, indicates that a comparison between two or more items, signals, etc., fails to provide the desired relationship.

As may be used herein, one or more claims may include, in a specific form of this generic form, the phrase “at least one of a, b, and c” or of this generic form “at least one of a, b, or c”, with more or less elements than “a”, “b”, and “c”. In either phrasing, the phrases are to be interpreted identically. In particular, “at least one of a, b, and c” is equivalent to “at least one of a, b, or c” and shall mean a, b, and/or c. As an example, it means: “a” only, “b” only, “c” only, “a” and “b”, “a” and “c”, “b” and “c”, and/or “a”, “b”, and “c”.

As may also be used herein, the terms “processing module”, “processing circuit”, “processor”, “processing circuitry”, and/or “processing unit” may be a single processing device or a plurality of processing devices. Such a processing device may be a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on hard coding of the circuitry and/or operational instructions. The processing module, module, processing circuit, processing circuitry, and/or processing unit may be, or further include, memory and/or an integrated memory element, which may be a single memory device, a plurality of memory devices, and/or embedded circuitry of another processing module, module, processing circuit, processing circuitry, and/or processing unit. Such a memory device may be a read-only memory, random access memory, volatile memory, non-volatile memory, static memory, dynamic memory, flash memory, cache memory, and/or any device that stores digital information. Note that if the processing module, module, processing circuit, processing circuitry, and/or processing unit includes more than one processing device, the processing devices may be centrally located (e.g., directly coupled together via a wired and/or wireless bus structure) or may be distributedly located (e.g., cloud computing via indirect coupling via a local area network and/or a wide area network). Further note that if the processing module, module, processing circuit, processing circuitry and/or processing unit implements one or more of its functions via a state machine, analog circuitry, digital circuitry, and/or logic circuitry, the memory and/or memory element storing the corresponding operational instructions may be embedded within, or external to, the circuitry comprising the state machine, analog circuitry, digital circuitry, and/or logic circuitry. Still further note that, the memory element may store, and the processing module, module, processing circuit, processing circuitry and/or processing unit executes, hard coded and/or operational instructions corresponding to at least some of the steps and/or functions illustrated in one or more of the Figures. Such a memory device or memory element can be included in an article of manufacture.

One or more embodiments have been described above with the aid of method steps illustrating the performance of specified functions and relationships thereof. The boundaries and sequence of these functional building blocks and method steps have been arbitrarily defined herein for convenience of description. Alternate boundaries and sequences can be defined so long as the specified functions and relationships are appropriately performed. Any such alternate boundaries or sequences are thus within the scope and spirit of the claims. Further, the boundaries of these functional building blocks have been arbitrarily defined for convenience of description. Alternate boundaries could be defined as long as the certain significant functions are appropriately performed. Similarly, flow diagram blocks may also have been arbitrarily defined herein to illustrate certain significant functionality.

To the extent used, the flow diagram block boundaries and sequence could have been defined otherwise and still perform the certain significant functionality. Such alternate definitions of both functional building blocks and flow diagram blocks and sequences are thus within the scope and spirit of the claims. One of average skill in the art will also recognize that the functional building blocks, and other illustrative blocks, modules and components herein, can be implemented as illustrated or by discrete components, application specific integrated circuits, processors executing appropriate software and the like or any combination thereof.

In addition, a flow diagram may include a “start” and/or “continue” indication. The “start” and “continue” indications reflect that the steps presented can optionally be incorporated in or otherwise used in conjunction with one or more other routines. In addition, a flow diagram may include an “end” and/or “continue” indication. The “end” and/or “continue” indications reflect that the steps presented can end as described and shown or optionally be incorporated in or otherwise used in conjunction with one or more other routines. In this context, “start” indicates the beginning of the first step presented and may be preceded by other activities not specifically shown. Further, the “continue” indication reflects that the steps presented may be performed multiple times and/or may be succeeded by other activities not specifically shown. Further, while a flow diagram indicates a particular ordering of steps, other orderings are likewise possible provided that the principles of causality are maintained.

The one or more embodiments are used herein to illustrate one or more aspects, one or more features, one or more concepts, and/or one or more examples. A physical embodiment of an apparatus, an article of manufacture, a machine, and/or of a process may include one or more of the aspects, features, concepts, examples, etc. described with reference to one or more of the embodiments discussed herein. Further, from figure to figure, the embodiments may incorporate the same or similarly named functions, steps, modules, etc. that may use the same or different reference numbers and, as such, the functions, steps, modules, etc. may be the same or similar functions, steps, modules, etc. or different ones.

Unless specifically stated to the contra, signals to, from, and/or between elements in a figure of any of the figures presented herein may be analog or digital, continuous time or discrete time, and single-ended or differential. For instance, if a signal path is shown as a single-ended path, it also represents a differential signal path. Similarly, if a signal path is shown as a differential path, it also represents a single-ended signal path. While one or more particular architectures are described herein, other architectures can likewise be implemented that use one or more data buses not expressly shown, direct connectivity between elements, and/or indirect coupling between other elements as recognized by one of average skill in the art.

The term “module” is used in the description of one or more of the embodiments. A module implements one or more functions via a device such as a processor or other processing device or other hardware that may include or operate in association with a memory that stores operational instructions. A module may operate independently and/or in conjunction with software and/or firmware. As also used herein, a module may contain one or more sub-modules, each of which may be one or more modules.

As may further be used herein, a computer readable memory includes one or more memory elements. A memory element may be a separate memory device, multiple memory devices, or a set of memory locations within a memory device. Such a memory device may be a read-only memory, random access memory, volatile memory, non-volatile memory, static memory, dynamic memory, flash memory, cache memory, and/or any device that stores digital information. The memory device may be in a form a solid-state memory, a hard drive memory, cloud memory, thumb drive, server memory, computing device memory, and/or other physical medium for storing digital information.

While particular combinations of various functions and features of the one or more embodiments have been expressly described herein, other combinations of these features and functions are likewise possible. The present disclosure is not limited by the particular examples disclosed herein and expressly incorporates these other combinations.

What is claimed is:

1. A mobile device comprises:
one or more interfaces;
one or more spectrometers operably coupled to an interface of the one or more interfaces, wherein each of the one or more spectrometers includes a plurality of spectral filters overlaying one or more optical sensors, wherein each of the one or more spectrometers has a sensing range within a predetermined range of optical wavelengths, wherein the one or more spectrometers is positioned in the mobile device to capture light radiation incident to the mobile device and wherein the one or more spectrometers are adapted to output information representative of captured light radiation;
a local memory;
a processing module operably coupled to the one or more interfaces and the local memory; wherein the processing module is adapted to receive the output information representative of captured light radiation and determine an accumulated light radiation for the mobile device;
a notification engine, wherein the notification engine is adapted to generate an alert when the accumulated light radiation exceeds a predetermined threshold.

2. The mobile device of claim 1, wherein the processing module is further adapted to determine an accumulated total radiation over a period of time T.

3. The mobile device of claim 2, wherein T includes a start time A, wherein A is based on manual entry by a user of the mobile device.

4. The mobile device of claim 3, wherein the time T includes a start time, wherein the start time A is based on a trigger event.

5. The mobile device of claim 4, wherein the trigger event is at least one of a location, a temperature, a temperature change, or a change in a rate of accumulation of measured light radiation.

6. The mobile device of one of claim 1, wherein the accumulated light radiation is determined over a spectrum of wavelengths.

7. The mobile device of claim 1, wherein the predetermined threshold is determined based on a comparison to a spectral profile of a set of spectral profiles.

8. The mobile device of claim 7, wherein the spectral profile is determined based on a user or a user type.

9. A method for determining exposure to light radiation, the method comprises:
sampling, using one or more spectral sensors associated with a mobile device, a received light spectrum, wherein each of the one or more spectral sensors includes a plurality of interference filters overlaying one or more optical sensors, wherein each of the one or more spectral sensors has a sensing range within a predetermined range of optical wavelengths, wherein the sensing range for the plurality of interference filters together including a spectrum of wavelengths;
outputting, by the one or more spectral sensors via one or more interfaces, information representative of the received light spectrum to one or more processing modules;
determining, by the one or more processing modules, based on the information representative of the received light spectrum, a light radiation level for at least a portion of the received light spectrum, wherein the determining a light radiation level is based on an accumulated light radiation at one or more wavelengths of the received light spectrum;
comparing, by the one or more processing modules, the light radiation level for at least a portion of the received light spectrum to a predetermined radiation threshold; and
when a predetermined light radiation threshold has been exceeded, generating an alert.

10. The method of claim 9, wherein the sampling is based on a snapshot of the received light spectrum at a time A.

11. The method of one of claim 9, wherein the predetermined light radiation threshold is based on ultraviolet light wavelengths.

12. The method of claim 9, wherein the accumulated light radiation is for light received over a time period T.

13. The method of one of claim 9, wherein the mobile device is adapted to transmit the alert to at least one of a display, a third party, a health provider or a conservator.

14. The method of one of claim 9, further comprises:
when the predetermined light radiation threshold has not been exceeded, predicting when the predetermined light radiation threshold will be exceeded based on a expected received light radiation and the accumulated light radiation.

15. The method of one of claim 14, wherein the expected received light radiation is based on at least one of a calculation of radiation received over a predetermined time, one or more historical patterns, time of day, season of year, activity level, activity type and user radiation mitigation.

16. The method of one of claim 9, further comprises:
when the predetermined light radiation threshold has not been exceeded, predicting when the predetermined light radiation threshold will be exceeded, wherein the predicting is based on a comparison of the received light radiation to a reference.

17. The method of one of claim 16, wherein the reference is at least one of a standardized radiation database, a radiation classification based on skin type for a user, a radiation classification based on skin sensitivity for the user, a prior classification for the user and a machine learning based classification.

18. The method of one of claim 16, wherein the reference is derived from crowdsourcing, wherein crowdsourcing includes light radiation data from a plurality of different users.

19. The method of one of claim 9, further comprises:
when the predetermined light radiation threshold has not been exceeded, transmitting a notification to a user associated with the mobile device.
